(19) United States
(12) Patent Application Publication
(10) Pub. No.: US 2026/0080982 A1
(43) Pub. Date: Mar. 19, 2026

(54) Systems and Methods for Generating Protocols Embodying Contrast and Radiation Dose Management Techniques

(71) Applicant: BAYER HEALTHCARE LLC, WHIPPANY, NJ (US)

(72) Inventors: Arthur Uber, III, Pittsburgh, PA (US); David Griffiths, Pittsburgh, PA (US); Linda van Roosmalen, Gibsonia, PA (US)

(21) Appl. No.: 19/109,553

(22) PCT Filed: Sep. 7, 2023
(86) PCT No.: PCT/US2023/032215

§ 371 Date: Mar. 6, 2025

Related U.S. Application Data
(60) Provisional application No. 63/374,979, filed on Sep. 8, 2022.

(51) Int. Cl. G16H 10/20; A61B 6/00; A61B 6/46; G16H 50/30

(52) U.S. Cl. CPC G16H 10/20; A61B 6/465; A61B 6/469; A61B 6/481; G16H 50/30

(57) ABSTRACT

A system and method for generating protocols that can be used for medical imaging studies. The method can include receiving information about a subject patient and information about a subject imaging study: determining, based on the information about the subject patient and the information about the subject imaging study, one or more risk factors particular to the subject patient: selecting two or more models, wherein the two or more models include at least one model of each of at least two aspects of the subject imaging study; and applying the two or more models to generate a baseline study protocol for the subject imaging study. The baseline study protocol is based upon at least the information about the subject patient and the one or more risk factors. The baseline study protocol includes parameters of the at least two aspects of the subject imaging study.

### CROSS REFERENCE TO RELATED APPLICATION

This application is a U.S. national phase application under 35 U.S.C. § 371 of PCT International Application No. PCT/US2023/032215, filed Sep. 7, 2023, which claims the benefit of U.S. Provisional Patent Application No. 63/374,979, filed Sep. 8, 2022, entitled “Systems and Methods for Generating Protocols Embodying Contrast and Radiation Dose Management Techniques,” the disclosures of which are hereby incorporated by reference in their entirety.

### BACKGROUND

### Field of the Disclosure

This disclosure relates to methods and techniques for selecting or generating study protocols for medical imaging procedures, as well as to methods and techniques for modifying or optimizing standard, initial, or baseline protocols for use for a particular patient and/or procedure in order to reduce risk to the patient, improve image quality, and/or improve workflow efficiency. Also provided are injection systems and fluid injectors that perform fluid injection procedures for a particular patient in accordance with a selected or modified patient protocol.

### Description of Related Art

In many medical diagnostic and therapeutic procedures, a medical practitioner, such as a medical technologist, injects a patient with one or more medical fluids. In recent years, a number of medical fluid delivery systems for pressurized injection of fluids, such as a contrast media (often referred to simply as “contrast”), flushing agent(s), such as saline, and other medical fluids, have been developed for use in imaging procedures such as angiography, computed tomography (CT), ultrasound, magnetic resonance imaging (MRI), positron emission tomography (PET), and other molecular imaging procedures such as single-photon emission tomography (SPECT) or hybrid modalities, such as PET/CT, PET/MRI, SPECT/CT, or SPECT/MRI. In general, these medical fluid delivery systems, such as powered fluid injectors, are designed to deliver fluids via one or more injection protocols. An injection protocol can include one or more injections, each comprising one or more phases to enhance regions of interest in a patient's body during diagnostic imaging. Examples of powered fluid injectors that are capable of delivering such fluids via user-programmable multi-phase injection protocols include the MEDRAD® Stellant CT Injection System and the MEDRAD® MRXperion MR Injection System, both of which are offered by Bayer HealthCare LLC.

There are several known risks and patient safety issues related to imaging procedures involving delivery of contrast and ionizing radiation to a patient. By way of example, issues of patient safety for contrast media injection can include one or more of the following: (i) extravasation prevention, detection, and minimization of extravasated substances; (ii) minimization of acute adverse events in each of contrast media naïve patients and patients with known atopy or a recorded acute adverse event due to a contrast media injection; (iii) prevention of a contrast media induced nephrotoxicity and/or a post contrast kidney injury; and/or (iv) management of patients to prevent a thyroid disorder, such as thyrotoxicosis (TX).

Extravasation can be an infrequent but significant problem in contrast enhanced medical imaging procedures, especially during nuclear medicine imaging procedures or when large volumes of contrast agent are involved. An extravasation occurs when contrast that is to be delivered to the central circulation through a peripheral vascular access instead enters the peripheral tissue (e.g., when contrast material escapes the vascular lumen and infiltrates the interstitial tissue during injection, etc.). The incidence of intravenous contrast material extravasation is typically reported as less than 1% and is not directly correlated with injection flow rate. However, some patients with extravasation may remain asymptomatic, while others may report swelling, tightness, stinging, or burning pain and may demonstrate edema, erythema, or tenderness at the injection site. Severe complications of extravasation include compartment syndrome, skin ulceration, and/or tissue necrosis.

Acute adverse events are dependent on applicated substances. The rate of acute adverse events for low osmolar iodinated contrast agents is approximately 0.2%-0.7%, and for severe acute reactions, 0.04%. The incidence of acute adverse events to gadolinium-based contrast agents (GBCAs) is low, occurring in approximately one in 10,000-40,000 injections. Most reactions are mild and transient, with skin reactions most frequently seen. Severe, life-threatening anaphylactoid reactions to gadolinium based contrast agents (GBCAs) are rare. Risk factors for acute adverse events to contrast agents may include previous reactions to iodinated contrast agents, severe allergies and reactions to medications and/or foods, a history of asthma, bronchospasm, and/or atopy, a history of cardiac or renal disease, and/or the like.

A contrast media induced nephrotoxicity may be defined as “a sudden deterioration in renal function (e.g., acute kidney injury, etc.) following a recent intravascular administration of contrast media in the absence of another nephrotoxic event”. Risk factors for a contrast media induced nephrotoxicity may include hypertension, proteinuria, gout, and/or previous renal surgery, or pre-existing chronic kidney disease (CKD). A risk for a contrast media induced nephrotoxicity is considered low in patients with normal, stable renal function. Similarly, a post-contrast acute kidney injury is a general term used to indicate a sudden deterioration in renal function within 48 hours of the intravascular administration of iodine-based contrast media.

In a case of iodinated contrast media application, which reflects the majority of contrast media usage, patients with untreated Graves' disease and/or multinodular goiter and thyroid autonomy, the elderly, and patients living in areas where dietary iodine deficiency is common may be at increased risk of thyrotoxicosis through excess iodine absorption. Moreover, the use of iodinated contrast agents before any planned radioactive iodine imaging or therapy may reduce the radioactive iodine uptake. In relation to risks from the contrast media atoms or molecules, it is the total dose, e.g. milligrams of iodine or gadolinium, not milliliters of fluid, which is relevant. The total milliliters (ml) of fluid delivered may present a risk of fluid overload for some compromised patients. Examples include patients on dialysis or those with congestive heart failure. In these patients the goal is to minimize the total amount of fluid, both contrast and saline flush, given to the patient.

For other imaging procedures, there are risks if the imaging procedure is not able to be performed in a timely way. For example, if a patient is presenting the symptoms of a stroke, rapid determination of the existence, type (hemorrhagic vs. ischemic), and extent of the stroke can have a significant impact on the survivability of the patient, i.e. “time is brain”. In this case, the priority is to obtain a sufficient diagnostic image as quickly as possible, and to use protocols that can be setup and performed efficiently and rapidly, though with potential tradeoffs in terms of contrast dose used, image quality, spatial resolution or other parameters. An additional risk is that the patient goes through the imaging study, receiving contrast and in some cases a radiation dose, and the study is non-diagnostic for one of many reasons, for example patient movement, poor timing between injection and study, the procedure itself being too complicated for the technologist to properly execute it, an imaging system that cannot perform the selected protocol as required, and many other causes.

Protocols for imaging procedures are developed in order to mitigate risks, avoid patient discomfort, improve image quality, and optimize workflow efficiency and costs. In general, protocols are developed by analyzing clinical trial data for past medical imaging procedures to identify possible changes for existing procedures that would improve compliance with guidelines and standards for patient safety. In order to assess efficacy of proposed changes for medical techniques, technologies, and standards, a technologist, such as a physician, must perform a procedure, use a new technology, and/or test a new standard with a number of patients in a number of multi-site clinical trials. Naturally, those trials must include a control group for proper assessment of the medical technique, technology, or standard. Following clinical trials, the technologist or physician typically describes and publishes his or her findings in a suitable medical journal. In addition, physicians may present their findings to peers at medical conferences. As can be readily understood, this process often may take a number of years. Moreover, the sheer magnitude of such undertakings often means that only the most deserving of medical techniques, technologies, and/or efforts to establish the most beneficial standards are pursued. In addition, enormous costs associated with studies prohibit most technologists and physicians from testing any techniques or equipment or from establishing new standards without assistance from large companies and research organizations, which have sufficient financial resources to fund these activities.

A medical injection protocol, which can be used for delivery of a drug, therapeutic agent, imaging agent, contrast, or another composition to a patient, can be identified, tested, and eventually adopted using this process of testing, publishing, and presenting described herein. For example, when performing a diagnostic evaluation that involves the use of a medical injector in combination with a scanning device (such as a CT (Computed Tomography) or MRI (Magnetic Resonance Imaging) scanner), it may be a widely accepted practice to inject contrast media into the patient at a standard rate, which is sufficient to provide images of sufficient quality for diagnostic evaluation for all or substantially all patients without exposing the standard patient to an unreasonable level of risk or patient discomfort. Examples can be found in the American College of Radiology Manual on Contrast Media. The standard rate of injection is generally determined or established using the clinical trial and testing methods described herein. Notably, a widely accepted “standard rate” would be expected to provide a sufficient level of enhancement for most or all patients, while, at the same time, resulting in no or a reasonably low level of risk for any patient.

Some physicians may adapt an accepted standard protocol to account for capabilities of new equipment or otherwise adapt protocols to changing circumstances to improve image quality and/or reduce risk to the patient. However, other practitioners, despite advances in technology, may continue to use the established protocol (e.g., an accepted contrast flow rate) simply because the flow rate falls within the standard established for the particular diagnostic technique. As discussed above, any efforts to modify or establish new standards can be time consuming and expensive. Accordingly, in some cases, existing standards used by physicians and technologists may not account for the most recent changes to technology or understanding about patient risks associated with medical imaging procedures.

There are known in the art various systems and methods that can algorithmically generate protocols for fluid injection (e.g., fluid injection protocols for a contrast procedure) based on inputs provided by a user or detected by system sensors. In particular, such protocol generation systems typically receive various inputs, such as patient information, desired enhancement, etc., and generate outputs in the form of parameters that can form part or all of a study protocol. In some cases, these algorithms could be used to update standard protocols to account for changes in technology or differences between specific types of medical equipment. However, existing models are generally based on a small number of input values and may not take into account the many sources of risk or patient discomfort, as well as the many ways in which image quality and/or workflow can be improved. Therefore, there is a need in the art for more robust and complete methods and techniques for modifying or optimizing medical imaging protocols, which address unique risks of a particular patient and confirm that images of sufficient quality can be obtained for the patient. The techniques, methods, and systems disclosed herein are provided to address these issues.

### SUMMARY

In some non-limiting aspects of this disclosure, provided is a system for generating protocols that can be used for medical imaging studies. The system includes one or more processors and non-transitory, computer readable media including instructions stored therein. The instructions, when executed by the one or more processors, will cause the system to: receive, from one or more data sources, information about a subject patient and information about a subject imaging study; determine, based on the information about the subject patient and the information about the subject imaging study, one or more risk factors particular to the subject patient; select two or more models, wherein the two or more models include at least one model of each of at least two aspects of the subject imaging study; and apply the two or more models to generate a baseline study protocol for the subject imaging study, wherein the baseline study protocol is based upon at least the information about the subject patient and the one or more risk factors, and wherein the baseline study protocol includes parameters of the at least two aspects of the subject imaging study.

In another non-limiting aspect of this disclosure, provided is a method for generating protocols that can be used for medical imaging studies. The method includes receiving, from one or more data sources, information about a subject patient and information about a subject imaging study; determining, based on the information about the subject patient and the information about the subject imaging study, one or more risk factors particular to the subject patient; selecting two or more models, wherein the two or more models include at least one model of each of at least two aspects of the subject imaging study; and applying the two or more models to generate a baseline study protocol for the subject imaging study, wherein the baseline study protocol is based upon at least the information about the subject patient and the one or more risk factors, and wherein the baseline study protocol includes parameters of the at least two aspects of the subject imaging study.

In another non-limiting aspect of this disclosure, provided is a method for generating protocols that can be used for medical imaging studies. The method includes receiving, from one or more data sources, information about a subject patient and a subject imaging study; generating a baseline study protocol based on the information about the subject patient and the subject imaging study; determining, based on the information about the subject patient and the subject imaging study, one or more risk factors particular to the subject patient; and modifying the baseline study protocol to address at least one of the one or more risk factors particular to the subject patient. Modifying the baseline study protocol to address the at least one of the one or more risk factors particular to the subject patient includes: performing an iterative process to optimize one or more parameters of the baseline study protocol to minimize the at least one of the one or more risk factors and generate a modified study protocol that will provide an image of sufficient diagnostic quality.

In another non-limiting aspect of the present disclosure, provided is a system for generating protocols that can be used for medical imaging studies. The system includes one or more processors and non-transitory, computer readable media including instructions stored therein. The instructions, when executed by the one or more processors, will cause the system to: receive, from one or more data sources, information about a subject patient and a subject imaging study; generate a baseline study protocol based on the information about the subject patient and the subject imaging study; determine, based on the information about the subject patient and the subject imaging study, one or more risk factors particular to the subject patient; and modify the baseline study protocol to address at least one of the one or more risk factors particular to the subject patient. Modifying the baseline study protocol to address the at least one of the one or more risk factors particular to the subject patient includes performing an iterative process to optimize one or more parameters of the baseline study protocol to minimize the at least one of the one or more risk factors and generate a modified study protocol that will provide an image of sufficient diagnostic quality.

Various aspects of the present disclosure may be further characterized by one or more of the following clauses:

Clause 1. A system for generating protocols that can be used for medical imaging studies, comprising: one or more processors; non-transitory, computer readable media comprising instructions stored therein, wherein the instructions, when executed by the one or more processors, will cause the system to: receive, from one or more data sources, information about a subject patient and information about a subject imaging study; determine, based on the information about the subject patient and the information about the subject imaging study, one or more risk factors particular to the subject patient; select two or more models, wherein the two or more models comprise at least one model of each of at least two aspects of the subject imaging study; and apply the two or more models to generate a baseline study protocol for the subject imaging study, wherein the baseline study protocol is based upon at least the information about the subject patient and the one or more risk factors, and wherein the baseline study protocol comprises parameters of the at least two aspects of the subject imaging study.

Clause 2. The system according to clause 1, wherein the baseline study protocol comprises a contrast injection protocol comprising at least a total contrast dose and a maximum flow rate, and an image acquisition protocol comprising at least scan parameters, scan duration, a timing parameter for coordination with a contrast injection, and one or more image reconstruction algorithms.

Clause 3. The system according to clause 1 or 2, wherein the two or more models comprise a plurality of models of one aspect of the imaging procedure, wherein the two or more models are configured to operate in parallel to transform a same or similar inputs.

Clause 4. The system according to any of the above clauses, wherein the two or more models are applied in a sequence, wherein the sequence is determined based upon at least one or more patient characteristics and a desired optimization of the one or more risk factors.

Clause 5. The system according to clause 4, wherein the system is configured to allow a user to accept or change the sequence based on the user's knowledge or preference.

Clause 6. The system according to any of the above clauses, wherein the instructions, when executed by the one or more processors, will additionally cause the system to: perform one or more iteration cycles through at least one of the two or more models to optimize one or more of the parameters of the baseline study protocol.

Clause 7. The system according to clause 6, wherein the instructions, when executed by the one or more processors, will additionally cause the system to: present to a user in a selectable format an outcome of one or more of the iteration cycles if none of the iteration cycles provides an optimized outcome.

Clause 8. The system according to any of the above clauses, further comprising a user interface, wherein expected parameters of the baseline study protocol are displayed for user confirmation or further adjustment.

Clause 9. The system of clause 8, wherein the user interface provides one or more selectable user interface elements which allows an operator to adjust the one or more risk factors particular to the subject patient.

Clause 10. The system of clause 9, wherein at least one of the one or more selectable user interface elements is in the form of a slider bar that is adjustable by a user.

Clause 11. The system of clause 10, wherein the user interface is a graphical user interface display screen, and the one or more user interface elements can be adjusted by a touch of the user on the graphical user interface display screen.

Clause 12. The system according to any of the above clauses, wherein at least one of the two or more models relates to at least one of a fluid injection aspect of the subject imaging study and an image creation aspect of the subject imaging study.

Clause 13. The system according to clause 12, wherein at least two of the two or more models relate to at least one of the fluid injection aspect of the subject imaging study and the image creation aspect of the subject imaging study.

Clause 14. The system according to clause 13, wherein a first of the two or more models relates to the fluid injection aspect of the subject imaging study and a second of the two or more models relates to the image creation aspect of the subject imaging study.

Clause 15. The system according to any of the above clauses, wherein the parameters include at least one of the following: total contrast volume, maximum flow rate, contrast delivery rate, average flow rate, contrast temperature, contrast viscosity, contrast concentration, IV access location, region of scan, potential applied to X-ray tube, maximum current applied to X-ray tube, scan speed, scan duration, radiation dose, signal/noise ratio, contrast/noise ratio, or spatial/resolution ratio.

Clause 16. The system according to any of the above clauses, wherein the information about the subject patient comprises at least one of height, weight, body mass index, cardiac output, gender, age, ethnicity, thoracic width, thoracic circumference, medications taken, underlying medical conditions, physical ability, vital signs, pregnant/expecting to become pregnant, genetic predisposition of the subject patient, allergies, results of previous imaging exams for the subject patient, and known radiation sensitivity of the subject patient.

Clause 17. The system according to any of the above clauses, wherein the one or more data sources comprise at least one of an electronic medical record (EMR) system comprising an electronic medical record of the patient, an electronic health record (EHR) system, a patient procedure tracking system, a radiology analytics system (RAS), a digital pathology system (DPS), a picture archive and communication system (PACS), a hospital data system, a hospital purchase order system comprising an order for a study to be performed for the subject patient, a database comprising previous scan results for the patient, a database comprising previous scan results for one or more other patients, or a government guidelines database of acceptable radiation dose and contrast dose levels.

Clause 18. The system according to any of the above clauses, wherein the information about the subject imaging study comprises information about a fluid injector associated with the subject imaging study wherein the information about the fluid injector includes information from a test injection or patency check using saline, information about capabilities and tolerances of the fluid injector, and/or presence of external sensors for monitoring injections performed by the fluid injector.

Clause 19. The system according to any of the above clauses, wherein the one or more risk factors are related to at least one of contrast dose, radiation dose, risk of extravasation, patient discomfort, risk of anaphylactic shock, and image quality.

Clause 20. The system according to clause 6, wherein the one or more iteration cycles optimize one or more parameters of the baseline study protocol by applying an algorithm that minimizes or maximizes selected parameter values, an algorithm for ensuring that certain parameters are within a target or threshold range, or a weighted function to parameter values of the baseline study protocol.

Clause 21. A method for generating protocols that can be used for medical imaging studies, comprising: receiving, from one or more data sources, information about a subject patient and information about a subject imaging study; determining, based on the information about the subject patient and the information about the subject imaging study, one or more risk factors particular to the subject patient; selecting two or more models, wherein the two or more models comprise at least one model of each of at least two aspects of the subject imaging study; and applying the two or more models to generate a baseline study protocol for the subject imaging study, wherein the baseline study protocol is based upon at least the information about the subject patient and the one or more risk factors, and wherein the baseline study protocol comprises parameters of the at least two aspects of the subject imaging study.

Clause 22. The method according to clause 21, wherein the baseline study protocol comprises a contrast injection protocol comprising at least a total contrast dose and a maximum flow rate, and an image acquisition protocol comprising at least scan parameters, scan duration, a timing parameter for coordination with a contrast injection, and one or more image reconstruction algorithms.

Clause 23. The method according to clause 21 or 22, wherein the two or more models comprise a plurality of models of one aspect of the imaging procedure, wherein the two or more models are configured to operate in parallel to transform a same or similar inputs.

Clause 24. The method according to any of clauses 21-23, wherein the two or more models are applied in a sequence, wherein the sequence is determined based upon at least one or more patient characteristics and a desired optimization of the one or more risk factors.

Clause 25. The method according to clause 24, wherein a user accepts or changes the sequence based on the user's knowledge or preference.

Clause 26. The method according to any of clauses 21-25, further comprising: performing one or more iteration cycles through at least one of the two or more models to optimize one or more of the parameters of the baseline study protocol.

Clause 27. The method according to clause 26, further comprising: presenting to a user in a selectable format an outcome of one or more of the iteration cycles if none of the iteration cycles provides an optimized outcome.

Clause 28. The method according to any of clauses 21-27, further comprising: displaying expected parameters of the baseline study protocol on a user interface for user confirmation or further adjustment.

Clause 29. The method according to any of clauses 21-28, wherein at least one of the two or more models relates to at least one of a fluid injection aspect of the subject imaging study and an image creation aspect of the subject imaging study.

Clause 30. The method according to clause 29, wherein at least two of the two or more models relate to at least one of the fluid injection aspect of the subject imaging study and the image creation aspect of the subject imaging study.

Clause 31. The method according to clause 30, wherein a first of the two or more models relates to the fluid injection aspect of the subject imaging study and a second of the two or more models relates to the image creation aspect of the subject imaging study.

Clause 32. The method according to any of clauses 21-31, further comprising: applying the baseline study protocol to perform the subject imaging study on the subject patient.

Clause 33. The method according to any of clauses 21-32, wherein the parameters include at least one of the following: total contrast volume, maximum flow rate, contrast delivery rate, average flow rate, contrast temperature, contrast viscosity, contrast concentration, IV access location, region of scan, potential applied to X-ray tube, maximum current applied to X-ray tube, scan speed, scan duration, radiation dose, signal/noise ratio, contrast/noise ratio, or spatial/resolution ratio.

Clause 34. The method according to any of clauses 21-33, wherein the information about the subject patient comprises at least one of height, weight, body mass index, cardiac output, gender, age, ethnicity, thoracic width, thoracic circumference, medications taken, underlying medical conditions, physical ability, vital signs, pregnant/expecting to become pregnant, genetic predisposition of the subject patient, allergies, results of previous imaging exams for the subject patient, and known radiation sensitivity of the subject patient.

Clause 35. The method according to any of clauses 21-34, wherein the one or more data sources comprise at least one of an electronic medical record (EMR) system comprising an electronic medical record of the patient, an electronic health record (EHR) system, a patient procedure tracking system, a radiology analytics system (RAS), a digital pathology system (DPS), a picture archive and communication system (PACS), a hospital data system, a hospital purchase order system comprising an order for a study to be performed for the subject patient, a database comprising previous scan results for the patient, a database comprising previous scan results for one or more other patients, or a government guidelines database of acceptable radiation dose and contrast dose levels.

Clause 36. The method according to any of clauses 21-35, wherein the information about the subject imaging study comprises information about a fluid injector associated with the subject imaging study wherein the information about the fluid injector includes information from a test injection or patency check using saline, information about capabilities and tolerances of the fluid injector, and/or presence of external sensors for monitoring injections performed by the fluid injector.

Clause 37. The method according to any of clauses 21-36, wherein the one or more risk factors are related to at least one of contrast dose, radiation dose, risk of extravasation, patient discomfort, risk of anaphylactic shock, or image quality.

Clause 38. The method according to clause 26, wherein the one or more iteration cycles optimize one or more parameters of the baseline study protocol by applying an algorithm that minimizes or maximizes selected parameter values, an algorithm for ensuring that certain parameters are within a target or threshold range, or a weighted function to parameter values of the baseline study protocol.

Clause 39. A method for generating protocols that can be used for medical imaging studies, comprising: receiving, from one or more data sources, information about a subject patient and a subject imaging study; generating a baseline study protocol based on the information about the subject patient and the subject imaging study; determining, based on the information about the subject patient and the subject imaging study, one or more risk factors particular to the subject patient; and modifying the baseline study protocol to address at least one of the one or more risk factors particular to the subject patient, wherein modifying the baseline study protocol to address the at least one of the one or more risk factors particular to the subject patient comprises: performing an iterative process to optimize one or more parameters of the baseline study protocol to minimize the at least one of the one or more risk factors and generate a modified study protocol that will provide an image of sufficient diagnostic quality.

Clause 40. The method of clause 39, wherein the baseline and modified study protocols comprise parameter values for at least one of the following: total contrast volume, maximum flow rate, contrast delivery rate, average flow rate, contrast temperature, contrast viscosity, contrast concentration, IV access location, region of scan, potential applied to X-ray tube, maximum current applied to X-ray tube, scan speed, scan duration, radiation dose, signal/noise ratio, contrast/noise ratio, or spatial/resolution ratio.

Clause 41. The method of clause 39 or 40, wherein the medical imaging studies comprise at least one of computed tomography (CT) imaging, magnetic resonance (MR) imaging, nuclear medicine, PET, SPECT, ultrasound, thermal imaging, infrared (IR) imaging, or combinations thereof.

Clause 42. The method of clause 41, wherein the combinations comprise a thermal/IR study; a PET/CT imaging study; a PET/MR imaging study; a SPECT/CT elastography study; or a study comprising optical and X-ray imaging.

Clause 43. The method of any of clauses 39-42, wherein the information about the subject patient comprises at least one of height, weight, body mass index, cardiac output, gender, age, ethnicity, thoracic width, thoracic circumference, medications taken, underlying medical conditions, physical ability, vital signs, pregnant/expecting to become pregnant, genetic predisposition of the subject patient, allergies, results of previous imaging exams for the subject patient, or known radiation sensitivity of the subject patient.

Clause 44. The method of any of clauses 39-43, wherein the information about the subject patient comprises radiation sensitivity of the subject patient and patient subject age.

Clause 45. The method of any of clauses 39-44, wherein the information about the subject patient comprises information representative of at least one of vascular access location, vein size, vein fragility, IV gauge, specific distances within body through vascular system of the subject patient, artery perfusion, or parenchyma.

Clause 46. The method of any of clauses 39-45, wherein the information about the subject patient comprises at least one physiological waveform of the subject patient.

Clause 47. The method of clause 46, wherein the at least one physiological waveform comprises an ECG waveform.

Clause 48. The method of any of clauses 39-47, wherein the one or more data sources comprises a caregiver's assessment of the subject patient.

Clause 49. The method of any of clauses 39-48, wherein the one or more data sources comprise at least one of an electronic medical record (EMR) system comprising an electronic medical record of the patient, an electronic health record (EHR) system, a patient procedure tracking system, a radiology analytics system (RAS), a digital pathology system (DPS), a picture archive and communication system (PACS), subject patient, a hospital data system, a hospital purchase order system comprising an order for a study to be performed for the subject patient, a database comprising previous scan results for the patient, a database comprising previous scan results for the one or more other patients, or a government guidelines database of acceptable radiation dose and contrast dose levels.

Clause 50. The method of any of clauses 39-49, further comprising performing a scout scan for the subject patient to determine study specific information, the study specific information comprising at least one of a length of scan region, scan time, length of plateau, and/or enhancement time for a bolus, and wherein the information about the subject patient comprises the study specific information determined by the scout scan.

Clause 51. The method of any of clauses 39-50, wherein the information about the subject imaging study comprises information about a fluid injector associated with the subject imaging study wherein the information about the fluid injector includes information from a test injection or patency check using saline, information about capabilities and tolerances of the fluid injector, and/or presence of external sensors for monitoring injections performed by the fluid injector.

Clause 52. The method of any of clauses 39-51, wherein some or all information about the subject patient is determined based on a question and answer session with the subject patient.

Clause 53. The method of any of clauses 39-52, wherein generating the baseline study protocol comprises applying a plurality of input parameters determined from the received information about the subject patient and the subject imaging study to at least one model, wherein the at least one model uses at least one of an algorithm, an attenuation and noise model, or patient fit and Monte Carlo simulation model for determining output parameters used for the baseline study protocol.

Clause 54. The method of clause 53, wherein the output parameters for the baseline study protocol comprise at least one of injection parameters; radiation dose outputs; or contrast dose information.

Clause 55. The method of clause 53, wherein generating the baseline study protocol comprises applying the plurality of input parameters to a first model and applying the output parameters from the first model to a second model to generate additional output parameters for the baseline study protocol.

Clause 56. The method of any of clauses 39-55, wherein the baseline study protocol is based on parameter values determined from at least one of a single model completed one time, a single model completed a single time selected by a user from a plurality of available models, multiple models completed in sequence, or a comparison of results for multiple models completed together.

Clause 57. The method of any of clauses 39-56, wherein the one or more risk factors are related to at least one of contrast dose, radiation dose, risk of extravasation, patient discomfort, risk of anaphylactic shock, or image quality.

Clause 58. The method of clause 57, wherein the radiation dose comprises a peak skin dose, an organ dose, breast dose (for female patients), effective dose, or cumulative dose.

Clause 59. The method of any of clauses 39-58, wherein the determination of the one or more risk factors particular to the subject patient is based on artificial intelligence using a model trained based on clinical data for patient outcome and image quality.

Clause 60. The method of any of clauses 39-59, wherein the iterative process to optimize the one or more parameters of the baseline study protocol comprises applying an algorithm that minimizes or maximizes selected parameter values, an algorithm for ensuring that certain parameters are within a target or threshold range, or a weighted function to parameter values of the baseline study protocol.

Clause 61. The method of any of clauses 39-60, wherein the iterative process to optimize the one or more parameters of the baseline study protocol is based on a comparison between predicted values for a dose for a past scan and an actual radiation dose for the past scan, any atypical events from previous scans for the subject patient, any atypical events from previous scans of a particular type, and/or optimization scores from previous scans.

Clause 62. The method of any of clauses 39-61, wherein modifying the baseline study protocol comprises adjusting a dose volume based, at least in part, on a measured gadolinium retention value for the subject patient.

Clause 63. The method of any of clauses 39-62, wherein modifying the baseline study protocol comprises at least one of reducing iodine concentration for a contrast media, reducing radiation dose, or changing an IV gauge for the medical imaging study.

Clause 64. The method of any of clauses 39-63, wherein performing the iterative process comprises optimizing a first parameter related to one of radiation dose, contrast dose, or risk of extravasation, and, after optimization of the first parameter, optimization of a second parameter related to another of radiation dose, contrast dose, or risk of extravasation.

Clause 65. The method of clause 64, wherein, following optimization of the second parameter, confirming that the modified study protocol comprising the first parameter and the second parameter provides the image of sufficient diagnostic quality.

Clause 66. The method of any of clauses 39-65, wherein determining whether the modified study protocol provides an image of sufficient diagnostic quality is based on a determination of whether image differentiation is sufficient based on at least one of tissue differentiation or Hounsfield units.

Clause 67. The method of clause 66, wherein the determination of whether the image is of sufficient diagnostic quality is based on a quantification of residuals and noise errors in the images generated by the modified study protocol.

Clause 68. The method of any of clauses 39-67, wherein the generated modified study protocol comprises a different protocol from the baseline study protocol performed on a same type of scanner as the baseline study protocol, use of different scanner and associated equipment to perform the modified study protocol, performing different modalities or tests compared to the baseline study protocol, and/or modification of one or more of the following fluid delivery parameters: concentration, flow rate, durations, iodine delivery rate, gadolinium delivery rate, dose volume, phase order, flow rate rise time, phase transition time, or injection delaying compared to the baseline study protocol.

Clause 69. The method of any of clauses 39-68, wherein the modified study protocol comprises a recommendation to increase hydration, sedation of the subject patient to reduce motion, and/or changing an IV location to an optimal IV location.

Clause 70. A system for generating protocols that can be used for medical imaging studies, comprising: one or more processors; non-transitory, computer readable media comprising instructions stored therein, wherein the instructions, when executed by the one or more processors, will cause the system to perform the method of any of clauses 39-69.

Clause 71. The system of clause 70, further comprising a user interface providing one or more selectable user interface elements which allows an operator to adjust the one or more risk factors particular to the subject patient.

Clause 72. The system of clause 71, wherein at least one of the one or more selectable user interface elements is in the form of a slider bar that is adjustable by the operator.

Clause 73. The system of clause 72, wherein the user interface is a graphical user interface display screen, and the one or more user interface elements can be adjusted by a touch of the operator on the graphical user interface display screen.

Clause 74. A fluid injector system for use in administering to a patient at least one fluid in a generated patient-specific protocol, the fluid injector system comprising: a control device operably associated with at least one drive component for use in pressurizing the at least one fluid through at least one disposable component into the patient; and the control device including at least one processor programmed or configured to enable programming of the patient specific protocol according to which the at least one drive component pressurizes the at least one fluid through the at least one disposable component into the patient so as to effect enhancement of at least one region of interest thereof over a scan duration of a diagnostic imaging procedure, wherein to generate the patient-specific protocol, the control device is further programmed or configured to: receive, from one or more data sources, information about the patient and an imaging study to be performed; generate a baseline study protocol based on the information about the patient and the imaging study to be performed; determine, based on the information about the patient and the imaging study to be performed, one or more risk factors particular to the patient; and modify the baseline study protocol to address at least one of the one or more risk factors particular to the patient, thereby providing the patient-specific protocol, wherein modifying the baseline study protocol to address at least one of the one or more risk factors particular to the patient comprises performing an iterative process to optimize one or more parameters of the baseline study protocol to minimize the at least one of the one or more risk factors and generate a modified study protocol that will provide an image of sufficient diagnostic quality.

### BRIEF DESCRIPTION OF THE DRAWINGS

FIG. 1A is a schematic drawing showing features of a model for generating a protocol for a medical imaging procedure, according to an aspect of the present disclosure;

FIG. 1B is a schematic drawing showing features of another exemplary model for generating a protocol for a medical imaging procedure, according to an aspect of the present disclosure;

FIG. 1C is a schematic drawing showing features of another exemplary model for generating a protocol for a medical imaging procedure, according to an aspect of the present disclosure;

FIG. 1D is a schematic drawing showing features of another exemplary model for generating a protocol for a medical imaging procedure, according to an aspect of the present disclosure;

FIG. 2A is a schematic drawing showing an exemplary model for generating a protocol for a medical imaging procedure that includes using two models in sequence to generate output parameters for the study protocol;

FIG. 2B is a schematic drawing of another exemplary model that obtains output parameters for a study protocol based on multiple models performed sequentially to generate a desired output, according to an aspect of the present disclosure;

FIGS. 3A-3C are schematic drawings showing trade-off considerations for optimizing a protocol for a CT imaging study, according to an aspect of the present disclosure;

FIGS. 3D and 3E are schematic drawings showing trade-off considerations for optimizing a protocol for an MR imaging study, according to an aspect of the present disclosure;

FIGS. 3F and 3G are schematic drawings showing trade-off considerations for optimizing a protocol for a Nuclear Medicine imaging study, according to an aspect of the present disclosure;

FIG. 3H shows a series of schematic graphs illustrating response curves resulting from an increase in an independent variable value;

FIGS. 4A and 4B are a flow chart showing a method for generating an optimized patient-specific protocol for a medical imaging study, according to an aspect of the present disclosure;

FIGS. 5A-1, 5A-2, 5B-1 and 5B-2 are tables of example optimizations for modifying a baseline study protocol for a particular patient, according to an aspect of the present disclosure;

FIGS. 5C, 5D-1 and 5D-2 are schematic drawings of user interfaces that can be used by a system operator for adjusting risk factors taken into consideration in generating a modified or optimized patient-specific study protocol, according to an aspect of the present disclosure;

FIG. 6A is a flow chart showing a method for generating an optimized patient-specific protocol for a medical imaging study for a first patient, according to an aspect of the present disclosure;

FIG. 6B is a flow chart showing a method for generating an optimized patient-specific protocol for a medical imaging study for a second patient, according to an aspect of the present disclosure;

FIG. 6C is a flow chart showing a method for generating an optimized patient-specific protocol for a medical imaging study for a third patient, according to an aspect of the present disclosure;

FIG. 6D is a flow chart showing a method for generating an optimized patient-specific protocol for a medical imaging study for a fourth patient, according to an aspect of the present disclosure;

FIG. 7 is a schematic drawing of a powered fluid injector system for fluid delivery to a patient in compliance with a patient-specific protocol generated by the methods and techniques disclosed herein;

FIG. 8A is a perspective view of another example of a powered fluid injector system, according to an aspect of the present disclosure;

FIG. 8B is a perspective view of the powered fluid injector system of FIG. 8A with an access panel in an open position;

FIG. 9 is a schematic view of various fluid paths within the powered fluid injector system of FIG. 8A;

FIG. 10 is a schematic drawing of an environment of use for a fluid injector system including a scan room and a control room, according to an aspect of the present disclosure;

FIG. 11 is a schematic drawing showing electronic components of a fluid injector system, according to an aspect of the present disclosure; and

FIG. 12 is a schematic drawing of an image creation system according to an aspect of the present disclosure.

### DETAILED DESCRIPTION

The illustrations generally show preferred and non-limiting examples or aspects of the systems and methods of the present disclosure. While the description presents various examples or aspects of the devices, it should not be interpreted in any way as limiting the disclosure. Furthermore, modifications, concepts, and applications of the disclosure's examples or aspects are to be interpreted by those skilled in the art as being encompassed by, but not limited to, the illustrations and descriptions herein.

The following description is provided to enable those skilled in the art to make and use the described examples or aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to an administration line, the term “proximal” refers to a portion of an administration line nearest to a powered fluid injector. When used in relation to an administration line, the term “distal” refers to a portion of an administration line nearest to an injection site on a patient. When used in relation to an administration line or a syringe of a powered fluid injector, the term “axial” refers to a direction along a longitudinal axis of a syringe or an administration line extending between the proximal and distal ends.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

It is also to be understood that the specific devices and processes illustrated in the attached drawings and described in the following specification are simply exemplary aspects of the disclosure. Hence, specific dimensions and other physical characteristics related to the examples disclosed herein are not to be considered as limiting.

With reference to the figures, the present disclosure is directed to techniques and methods for generating a fluid injection study protocol and/or an imaging study protocol for a medical imaging procedure. In particular, systems and/or methods disclosed herein can be used for generating procedure protocols (e.g., sets of parameters that can be used by injectors, scanners, or other modalities to complete an imaging procedure). Desirably, generated or optimized patient protocols provide at least acceptable (“good enough”) imaging results (e.g., diagnostic images) every time for every patient. As described in detail herein, the generated or optimized study protocols can be based on data from many sources including patient specific data, as well as data collected from previous imaging procedures, other patients, and/or data for a patient population. Input data can be analyzed using numerous mathematical models for determining or modifying a protocol to produce an optimized patient specific protocol. Furthermore, the study protocol optimization methods and techniques disclosed herein should be easy to implement with minimal work for the technologist and at a minimal cost/harm to the patient and the healthcare system.

As used herein, a “protocol” or “study protocol” can refer to the sequence of events that occur during an imaging procedure, as well as to multiple input parameters for medical devices, such as a fluid injector and image scanner, used during the imagining procedure. For example, the study protocol can include settings or parameters related to a contrast being used, such as a total contrast volume, flow rate (e.g., contrast delivery rate), and/or contrast dose temperature, which is related to viscosity of a contrast medium. Parameters of an injection protocol can also be related to a radiation dose or scanner setting, such as current (e.g., current passed through X-ray tube in milliamperes (mAs)) or potential applied to the X-ray tube, which can be measured as the kilovoltage peak (kVp). Other scan parameters can include scan speed, scan duration, beam width, slice width, and radiation dose. Parameters of an imaging procedure can also be related to image quality for images obtained during the study or procedure. For example, parameters of an imaging protocol can include signal-to-noise (S/N) ratio, contrast-to-noise (C/N) ratio, and/or spatial resolution for images captured according to the protocol. Other relevant protocol parameters can include maximum injection rate (e.g., to address extravasation risk), injection location (e.g., venous or arterial), bolus shape, contrast temperature, contrast viscosity, contrast concentration, image resolution, etc.

A “protocol” for an imaging procedure being performed can include selected values for one or more of these parameters, as well as any other parameters believed to be relevant to a fluid delivery and imaging procedure being performed. As described in further detail herein, a study protocol can be generated for a normal or average patient or modest number of patients who span the normal distribution of patients. This normal or average patient protocol (also referred to herein as an initial or baseline protocol) is desirably sufficient to provide a reasonable quality image for the average patient without exposing the patient to an unreasonable risk from radiation, contrast, or any of the other previously described sources of risk. For example, at a talk at the Radiological Society of North America's 2016 annual meeting, Dominik Fleischman recommends that a 64 slice CT Angiogram (CTA) use a scan time of 10 seconds, an injection duration of 18 seconds, and contrast flow rates and volumes that are adjusted for patient weight. Patients less than 55 kg receive 72 ml, patients 55-65 kg receive 81 ml, patients 66-85 kg receive 90 ml, patients 86-95 kg receive 99 ml, and patients >95 kg receive 108 ml. The length of the scan is adjusted for each patient based on their tomogram (the CT scout image). This is an example of a modest number of initial protocols. The methods and techniques disclosed herein are provided to optimize or customize the generated or initial protocol for a particular patient based, for example, on an assessment of patient-specific risks, to improve image quality and/or reduce risks for the patient. The optimization or customization can be based on specific features of the patient, medical devices being used, and/or procedures being performed. Thus, in summary, the systems and methods described herein are intended to provide mechanisms by which an operator can optimize certain study parameters in order to fit the study protocol to the specific patient and to address any risk factors that may exist for the particular patient. In addition, while the examples and many embodiments below are focused on contrast-related studies, such as CT and MRI, the methods and systems of the present disclosure for using models and iterating around multiple models can be adapted to non-contrast studies as well, including studies in which radiation dose, image quality, risk of motion, and claustrophobia still apply.

By way of example, parameters related to contrast delivery to the patient can be adjusted to improve patient safety and effectiveness of a scanner used for a particular imaging procedure. For example, conventional fluid injectors generally eject fluid at a high rate to ensure that a sufficient fluid dose is delivered to a patient within a reasonable period of time. However, for some patients and procedures, the rate of injection of contrast media may not need to be as high as the rate initially thought. For example, slight variations in scanning technology, patient demographics, patient physiology, or other protocol measures may allow for an optimal result to be achieved using less contrast media for some patients and procedures. Also, the sensitivity of the scanner used for a particular diagnostic may have improved (and probably has improved) since the development of the standard(s) (e.g., the standard protocol for the normal patient) associated with its use. By way of another example, the model patient for which a particular standard (e.g., a particular standard or baseline protocol) was developed may vary slightly in height or weight from the patient now subject to the imaging procedure. Therefore, contrast injection rate and/or volume can be adjusted to account for these minor differences between patients and scanner devices.

The present inventors have recognized many benefits of reducing contrast dose. For example, modifying contrast injection rate and/or contrast volume can reduce procedure costs by reducing an amount of a contrast media that is injected to a patient. Reducing a contrast dose lessens the risk that a patient will have an adverse reaction to the contrast media. In addition, due to its increased sensitivity of some modern scanners, such scanners may perform optimally at a lower injection rate than is envisioned by current standard protocols. In such instances, scanner performance may actually be hindered by following a standard protocol in which contrast is injected at the conventional high flow rate. Many practitioners may also be unaware of improvements realized by other practitioners who have successfully achieved optimal study results using protocols that diverge from what, at one time, was the accepted practice or which are more closely tailored to the actual study being performed. Accordingly, making adjustments to standard injection protocols and parameter values is beneficial for improving patient outcomes.

In some examples, the methods and systems of the present disclosure are intended to act as a “guidance system” in the form of an overall image creation system model that relies upon two or more sub-models of various aspects or subsystems of the overall imaging system, for example an injector and an imaging device, to generate the patient specific protocol. In such instances, the systems and methods disclosed herein may include multiple sub-models of the same or overlapping aspects of an image creation system based upon different levels of detail, variables, or experimental results. The image creation system may also involve the patient, the technologist, and optionally the radiologist as sources of data and/or feedback. Furthermore, the performance of this overall image creation system model can be simulated based upon user guided inputs and existing or missing input data to converge on a suggested optimized or patient-specific protocol or protocols. Because of the high likelihood of missing data and an incorrect model direction (e.g., a variable to optimize is a model input when the model cannot be explicitly inverted), the overall performance may be iterated to converge on the recommended, modified, or adapted study protocol(s).

Thus, in some examples, the systems and methods of the present disclosure arrive at the modified or customized protocol by an iterative optimization method. For example, methods and systems disclosed herein can apply patient and study data (e.g., patient demographics, patient study history, information about study type), risk assessments (e.g., extravasation risk, radiation risk, contrast risk), and protocol computational guidance (e.g., existing protocol algorithms and methods) to an iterative process in order to generate study protocols capable of producing a diagnostic quality image with sufficient image contrast (e.g., tissue differentiation, Hounsfield units, etc.) and low enough noise (e.g., detector noise), while, at the same time, avoiding or minimizing particular risk factors of the patient. In particular, an object of the present disclosure is to provide a system and method that perform various optimizations in a system and/or algorithm to permit an automatic tradeoff between optimization tasks in order to simplify and expedite operation of imaging procedures.

In some examples, the methods and systems of the present disclosure rely upon inputs that are independent or non-controllable (e.g., inputs that do not change during a procedure). Such inputs can include, for example, patient information, procedure information, disease screening information, equipment information, etc. Examples of patient information can include patient physical or anthropometric information (e.g., height, weight, lean body mass, gender, age, race, thoracic width, thoracic diameter, body mass index, body surface area, waist to hip ratio, metal or other implants, pregnancy, etc.), patient physiological information (e.g., allergies, cardiac output, ejection fraction, blood pressure, blood flow velocity, pulse rate, flow rate capability due to vein size, respiration rate, glucose level, estimated glomerular filtration rate, serum creatinine level, coagulation status, blood oxygen saturation, vascular access location), patient history (e.g., results from previous imaging exams, prior contrast and radiation dose received, occupational radiation dose estimate, claustrophobia, history of uncooperative behavior), and operator assessment of the patient (e.g., breath holding ability, ability to follow directions, need for sedation, critical/stable/ambulatory status, fast/non-fast). Examples of procedure information can include procedure type (e.g., first pass, arterial phase, parenchymal phase, venous phase, cardiac, abdomen, neuro, peripheral, Computed Tomography Angiography (CTA) vs. regular CT, lung cancer vs. pulmonary embolism, target tissue to be imaged, presence of contrast, emergency vs. routine, etc.), scan volume, procedure goals (e.g., screening vs. diagnostic, assessing treatment, monitoring follow up, biopsy, etc.), procedure history (e.g., previous predictions vs. actual radiation dose for this scan of this patient or of this study, any atypical events from previous scans of this type or of this patient, benchmarked comparables/data, prospective or retrospective gating, etc.), type of scan planned (e.g., modality, full scan details, length covered and duration, kVp, multi-spectral imaging, multi-modal imaging scout scan), and contrast test bolus data (e.g., flow and pressure to estimate impedance, test bolus curve, regions of interest, phantom data). Examples of equipment information can include sensor availability, equipment type, equipment quality, equipment age, and/or cost to use the equipment. Sources for independent inputs are not necessarily limited to the types of data disclosed herein. Other non-limiting sources of information or inputs for optimizing or customizing a patient protocol can be, for example, a paper order from the patient's caregiver, a verbal (Q&A) assessment of the patient, written guidelines for the procedure being performed, facility rules and guidelines for the procedure, hospital information systems, or a patient's chart and lab work.

With continued reference to the figures, the present disclosure is also directed to a contrast injector system and a controller configured to cause a fluid injector of the system to expel fluid (e.g., a contrast agent or saline) at a predetermined or calculated rate and/or pressure in accordance with the baseline and/or customized protocols generated by the methods and techniques disclosed herein. The hardware associated with the system can include, for example, one or more scanning devices (e.g., a CT (Computed Tomography) or MRI (Magnetic Resonance Imaging) scanner), one or more medical injectors (e.g., contrast injectors, pharmacologic stress agent injectors), one or more databases to store the information and generated protocols discussed herein, and one or more processors (e.g., computers) configured to execute programming instructions stored in non-transitory computer media in order to perform the tasks and functions described herein.

### Baseline or Conventional Protocol Determination

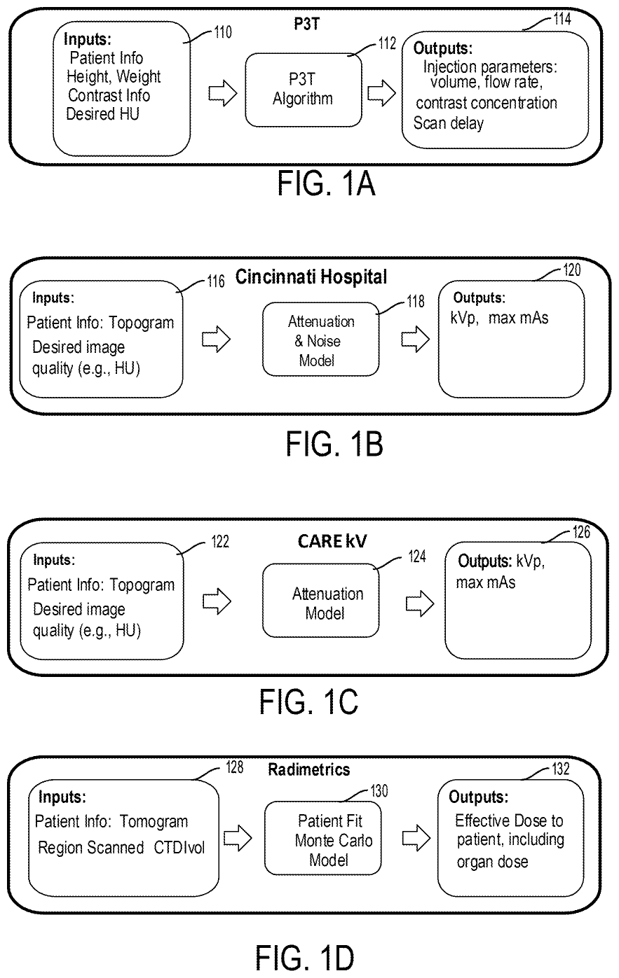

The methods and techniques disclosed herein generally begin by generating or providing a standard or baseline protocol for a normal patient (e.g., a patient of average height, weight, age, etc.) and/or of a normal patient of a specified gender, age, and/or general size (e.g., small, medium, or large). In some examples, the normal or conventional protocol can be a model that (e.g., based on long term and widespread use) is known to provide a reasonable radiation dose and to obtain images of reasonable quality for an otherwise healthy patient. The model can use standard patient parameters as inputs for the model, including patient size, weight, and gender. Based on such common or general inputs, a protocol can be generated which, desirably, should work for any patient to provide reasonable images (e.g., images of sufficient quality to use for diagnosis or as a record of a patient's current condition). FIGS. 1A-1D are schematic drawings showing several of these conventional models for generating baseline protocols for an imaging procedure being performed.

A model has as input one or more model inputs and provides one or more model outputs. Some of the model inputs may be about the imaging study. Among the model outputs are aspects of injector and/or scanner protocols and/or information about what is expected to happen, such as contrast dose to be used or radiation dose expected to be received by the patients. For example, the current P3T algorithm outputs an injector protocol and timing information to synchronize between the injector and the scanner and the current Siemens CARE kV model outputs CT scan parameters.

Some models are not invertible, meaning that an inverse model cannot be created, so the original model inputs cannot be determined from the original model outputs. An example of this is the Ty Bae model for flow of contrast through the body, which, given a specific contrast injection bolus as input, produces the time varying concentration of contrast concentration in various body parts as the output. If the goal is to determine the injection bolus for a specific patient to achieve a specific organ image enhancement, this cannot easily be done because all of the parameters of the model are not known for the specific patient.

One way around this is to create a reasonable model based on the patient's height, weight, and cardiac state, if known. The model is given a reasonable bolus as input and the output is computed. By successively adjusting the bolus length and re-computing the output, it is possible to achieve the desire length of image enhancement. This is discussed in U.S. Patent Application Publication No. 2019/0012932 A1 titled “Simulator, Injection Device Or Imaging System Provided With Simulator, And Simulation Program,” which is incorporated herein by reference.

Another approach is to pre-perform at least some of iterations, for example to create a data set or “dictionary” or “library” of a large but finite number of input combinations and their resulting output sets. This approach has been done for MRI signal evolution as part of the process called Magnetic Resonance Fingerprinting (MRF) as described in U.S. Pat. No. 8,723,518 titled “Nuclear Magnetic Resonance (NMR) Fingerprinting,” and U.S. Patent Application Publication No. 2014/0167754 A1 titled “Magnetic Resonance Fingerprinting (MRF) With Echo Splitting,” both of which incorporated herein by reference herein. Then, for a given desired output set, it can be compared to the data sets in the dictionary, for example using Orthogonal Matching Pursuit (OMP), and when the closest match is found, the related set of inputs provides the parameters to be used for the bolus design and/or scan design, optionally with interpolation between the nearest dictionary entries and optional subsequent confirmation by the model using the interpolated parameters. Optionally, further small adjustments can be made through iteration, although this likely is not necessary or worth the time or effort.

Each of the models described herein encompasses certain aspects of the overall imaging procedure. Historically, separate models have related to the injector device and to the scanning device which are two aspects of the imaging procedure. Thus, the systems and methods described herein optionally use multiple models to optimize the overall imaging procedure. It is anticipated that new models will be developed which may cover more or fewer aspects than those described herein. For example, within the imaging aspect for a CT scan, there is the CT scan design, radiation dose estimation, and image quality estimation which generally are different models but may be encompassed as a single model or a family of connected or interoperable models. There may also be specific models for specific studies as is the case for the P3T algorithms of Bayer Healthcare LLC or the FAST CARE technology of Siemens as listed at https://www.siemens-healthineers.com/it/computed-tomography/technologies-innovations/fast-care. These more encompassing models may be used as part of this disclosure with the potential benefits of faster protocol development, more customization and precision of the protocol to the patient, and reduced overall risk to the patient. Optional additional aspects of the imaging procedure may include patient handling and care before, during and after, including risk mitigations suggested by the optimization workstation before, during and after, the overall workflow such as patient and machine scheduling, and post processing of the data acquired by the scanning device. These are relevant as it relates to optimizing the overall protocol used for a patient with a particular risk profile.

In further aspects of this disclosure, more sophisticated or encompassing models may be built. For example, CARE kV models may be developed that include both a low contrast dose and a normal contrast dose, with the low contrast dose providing, for example, a lower kVp to increase iodine sensitivity at the cost of a higher mAs and thus a little bit higher radiation dose. By having these multiple models that incorporate multiple risk factors as inputs, it may be possible to reduce the amount of iteration that is required to achieve a satisfactory optimized imaging protocol.

One approach to building a more encompassing model is to record the optimized protocols that are created, making a database of patient and optimized imaging protocols. This database preferably also captures the actual results, such as image quality or the time course of enhancement and compares them to the results predicted by the model(s) used. As this database is built up, when a new patient arrives, the database may first be scanned to check for a match. If such a match exists, that protocol may be used for the present patient. Alternatively, as computer power increases and storage costs decrease, it will be possible to, over some time, model a wide range of patients and compute their optimized protocols and store them in a large database. This database can then be queried when a new patient arrives for the nearest matching protocol which is used for their imaging protocol. Thus, the modeling process does not have to be done real time for each patient but can be done ahead of time, in whole or in part, to streamline the modeling process required to find an optimized protocol. In some systems, optimization may include a database look up in a database of optimized imaging protocols.

For example, FIG. 1A is a schematic drawing showing, at a high level, a CT dosing system by Bayer HealthCare LLC, referred to as the P3TR software system, which can provide CT dosing recommendations for various studies, including cardiac, pulmonary angiography, and abdomen. As shown in FIG. 1A, at box 110, the P3T system receives the following inputs: patient information (e.g., patient weight and height), contrast information (e.g., concentration), and desired imaging characteristics (e.g., Hounsfield units). The model uses an algorithm, at box 112, to generate outputs for an injection. Specifically, as shown in box 114, the output parameters can include volume, flow rate, contrast concentration, as well as scan delay. Further information about the P3TR software system can be found in U.S. Pat. Nos. 7,925,330, 8,428,694, and 10,166,326, which are incorporated herein by reference.

FIG. 1B shows another model for determining a protocol, referred to as the Cincinnati Hospital model. As shown in box 116, inputs for the model include patient information (e.g., patient height, weight, and gender) and a numerical value representative of a desired image quality. The model applies an attenuation and noise model (shown in box 118) to generate output parameter values for radiation dose (kilovoltage peak (kVp) and milliamperes (mAs)), which are shown in box 120. Further information about the Cincinnati Hospital model can be found in U.S. Patent Application Publication No. 2014/0270053A1 titled “Method for Consistent and Verifiable Optimization of Computed Tomography (CT) Radiation Dose”, which is incorporated herein by reference.

FIG. 1C is a schematic drawing showing high level features of another model, referred to as the CARE kV system by Siemens. As shown in box 122, the CARE kV system receives inputs in the form of patient information from a tomogram and an input value for a desired or required image quality. The image quality value can be a value for image differentiation or Hounsfield units. Other values for image quality can include a desired signal-to-noise or contrast-to-noise ratio. At box 124, an attenuation model is applied to the input values, which generates scan parameters including a value for kVp and max mAs, as shown at box 126. Further information about the CARE kV system can be found in the document titled “How to Scan with CARE kV”, available online at https://cdn0.scrvt.com/39b415fb07de4d9656c7b516d8e2d907/1800000000073220/c2ab5e6cbb6e/CT_How_to_reduce_dose_CARE_kV_final_1800000000073220.pdf, which is incorporated by reference herein.

FIG. 1D is a schematic drawing showing a protocol for the Radimetrics® Enterprise Application, a radiation dose management platform from Bayer HealthCare LLC. As shown in box 128, the model uses inputs including patient information (e.g., from a tomogram), along with a desired CTD/vol for a region to be scanned. At box 130, patient fit for the input information is identified by a Monte Carlo model or simulation. At box 132, the model provides output values for effective dose to patient and/or effective dose to an organ of the patient. Further information about the Radimetrics radiation dose management platform can be found in U.S. Pat. No. 10,438,348, assigned to Bayer Healthcare LLC, which is incorporated herein by reference. Radimetrics is designed as a dose calculation system to be used typically after a CT scan so that the organ dose of a patient may be determined and recorded. In this invention, it may be used as one of the two or more models in the iterative optimization system described herein.

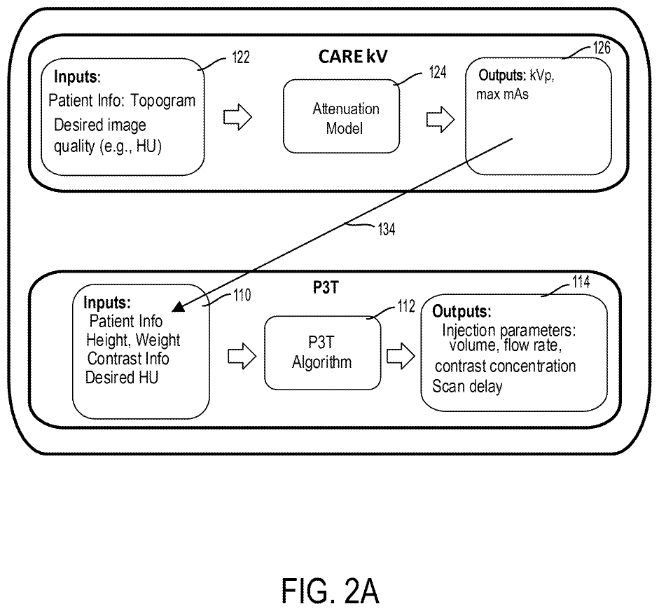

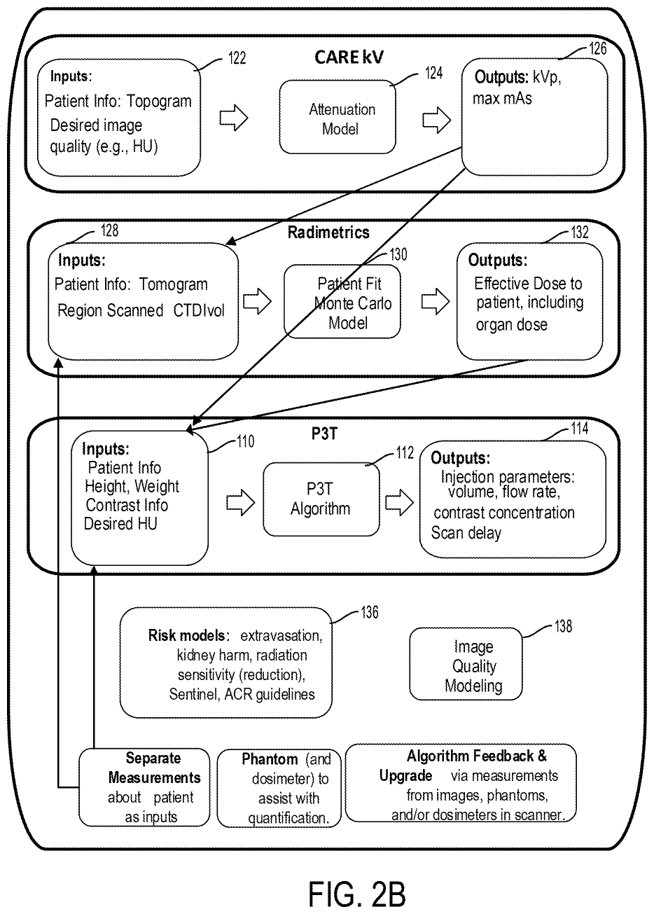

In some examples, an initial or baseline protocol can be determined using only one model. For example, a system can be configured to use only one of these models or any other known model for generating an initial or baseline protocol for a patient. As described in further detail herein, the initial or baseline protocol can then be adjusted, modified, or optimized to provide the patient-specific protocol. In other examples, the system can be configured to receive a user selection about which model to use. For example, the user may select an initial model to use to determine the baseline protocol parameters by, for example, selecting one specific model from a user interface screen listing multiple available models. In still other examples, a system or method can be configured to determine different protocols or output values using multiple different models. The system operator (or the system itself) can review the outputs generated by the different models and select which model and/or generated outputs to use as an initial or baseline protocol with the choice depending upon the various risk of concern for that patient. In other examples, as shown in FIGS. 2A and 2B, multiple models can be used together for generating output values or parameters for the initial or baseline protocol. The models discussed herein are not intended to be limited, and additional models can be developed as knowledge and/or technology improves. The models can also be customized per the policies and/or guidelines of a particular institution (e.g., hospital, healthcare system, or country) in which they are used. A database can be used to store the models, and the database can reside in the cloud.

In particular, as illustrated in FIGS. 2A and 2B, it is also possible to utilize certain models in sequence or combination to provide outputs for an initial or baseline protocol. For example, as shown in FIG. 2A, the CARE KV model (from FIG. 1C) can be used to determine outputs for kVp and max mAs, as shown in box 126. As shown by line 134, the values for kVp and max mAs can then be used as inputs for the P3T model (from FIG. 1A). As previously described, the P3T model can then be used to determine output parameters including injection parameters, contrast information, and scan delay, as shown in box 114.

FIG. 2B is a schematic drawing showing a more complex arrangement in which aspects and concepts of the CARE kV model (from FIG. 1C), the Radimetrics model (from FIG. 1D), the P3T model (FIG. 1A), and other models, such as risk models (box 136) for extravasation, kidney harm, radiation, sensitivity, and/or American College of Radiology (ACR) guidelines and/or image quality modeling (box 138) are considered together. Beneficially, the different models can use separate patient information measurements (e.g., the Radimetrics model uses a tomogram and the P3T model uses patient height and weight), optionally in combination with outputs from one or more of the other models, to provide a more complete indication of patient size and physical features. Patient function information, such as cardiac output, may also be used as an input for one or more of the models. As shown in FIG. 2B, outputs from the CARE kV model (box 126) related to kVp and max mAs can be used as inputs for Radimetrics model (box 128 in FIG. 1D) and the P3T model (box 110 in FIG. 1A). The models can be iteratively performed, while taking into account the risk models (box 136) and/or image quality models (box 138), to account for trade-offs between, for example, radiation dose, contrast (e.g., iodine) dose, and/or image quality. As shown at box 114, outputs from different iterations of the P3T model, such as injection parameters, contrast parameters, and scan delay parameters, can be used for the initial or baseline protocol.

Other models, which can also be adapted for use with the methods and systems of the present disclosure, have also attempted to provide quantitative analysis of the injection process during CT angiography (CTA) to improve and predict arterial enhancement. For example, Bae and coworkers developed pharmacokinetic (PK) models of the contrast behavior and solved the coupled differential equation system with the aim of finding a driving function that causes the most uniform arterial enhancement. See K. T. Bae, J. P. Heiken, and J. A. Brink, “Aortic and hepatic contrast medium enhancement at CT. Part I. Prediction with a computer model,” Radiology, vol. 207, pp. 647-55 (1998); K. T. Bae, “Peak contrast enhancement in CT and MR angiography: when does it occur and why? Pharmacokinetic study in a porcine model,” Radiology, vol. 227, pp. 809-16 (2003); K. T. Bae et al., “Multiphasic Injection Method for Uniform Prolonged Vascular Enhancement at CT Angiography: Pharmacokinetic Analysis and Experimental Porcine Method,” Radiology, vol. 216, pp. 872-880 (2000); U.S. Pat. Nos. 5,583,902, 5,687,208, 6,055,985, 6,470,889 and 6,635,030, the disclosures of which are incorporated herein by reference. An inverse solution to a set of differential equations of a simplified compartmental model set forth by Bae et al. indicates that an exponentially decreasing flow rate of contrast medium may result in optimal/constant enhancement in a CT imaging procedure. However, the injection profiles computed by inverse solution of the PK model are profiles not readily realizable by most CT power injectors without major modification.

In another approach, Fleischmann and coworkers treated the cardiovascular physiology and contrast kinetics as a “black box” and determined its impulse response by forcing the system with a short bolus of contrast (approximating a unit impulse). In that method, one performs a Fourier transform on the impulse response and manipulates this transfer function estimate to determine an estimate of a more optimal injection trajectory than practiced previously. See D. Fleischmann and K. Hittmair, “Mathematical analysis of arterial enhancement and optimization of bolus geometry for CT angiography using the discrete Fourier transform,” J Comput Assist Tomogr, vol. 23, pp. 474-84 (1999), the disclosure of which is incorporated herein by reference.

Uniphasic administration of contrast agent (typically, 100 to 150 mL of contrast at one flow rate) results in a non-uniform enhancement curve. See, for example, D. Fleischmann and K. Hittmair, supra; and K. T. Bae, “Peak contrast enhancement in CT and MR angiography: when does it occur and why? Pharmacokinetic study in a porcine model,” Radiology, vol. 227, pp. 809-16 (2003), the disclosures of which are incorporated herein by reference. Fleischmann and Hittmair thus presented a scheme that attempted to adapt the administration of contrast agent into a biphasic injection tailored to the individual patient with the intent of optimizing imaging of the aorta. A fundamental difficulty with controlling the presentation of CT contrast agent is that a hyperosmolar drug diffuses quickly from the central blood compartment. Additionally, the contrast is mixed with and diluted by blood that does not contain contrast.

Fleischmann proscribed that a small bolus injection, a test bolus injection, of contrast agent (16 ml of contrast at 4 ml/s) be injected prior to the diagnostic scan. A dynamic enhancement scan was made across a vessel of interest. The resulting processed scan data (test scan) was interpreted as the impulse response of the patient/contrast medium system. Fleischmann derived the Fourier transform of the patient transfer function by dividing the Fourier transform of the test scan by the Fourier transform of the test injection. Assuming the system was a linear time invariant (LTI) system and that the desired output time domain signal was known (a flat diagnostic scan at a predefined enhancement level), Fleischmann derived an input time signal by dividing the frequency domain representations of the desired output by that of the patient transfer function. Because the method of Fleischmann et al. computes input signals that are not realizable in reality as a result of injection system limitations (for example, flow rate limitations), one must truncate and approximate the computed continuous time signal.

### Refining or Optimizing a Protocol

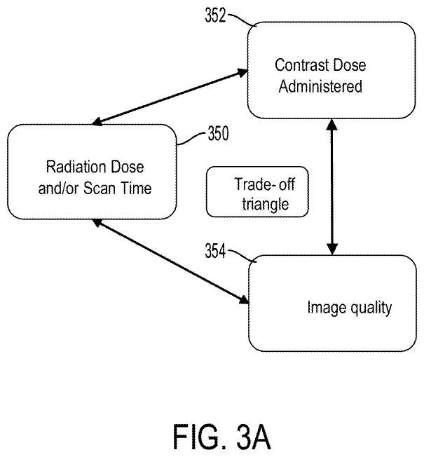

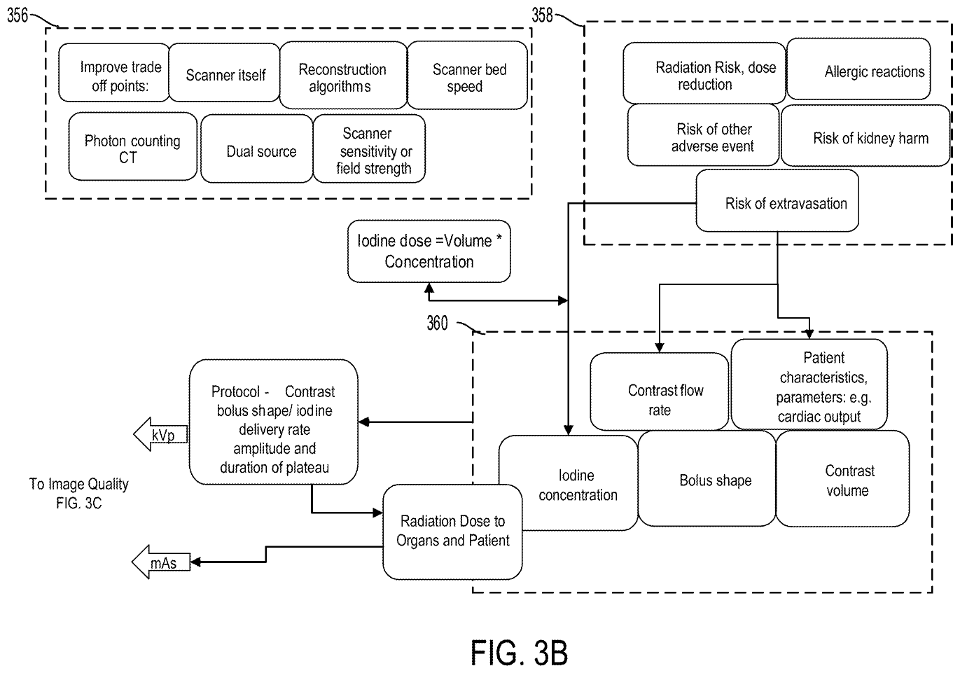

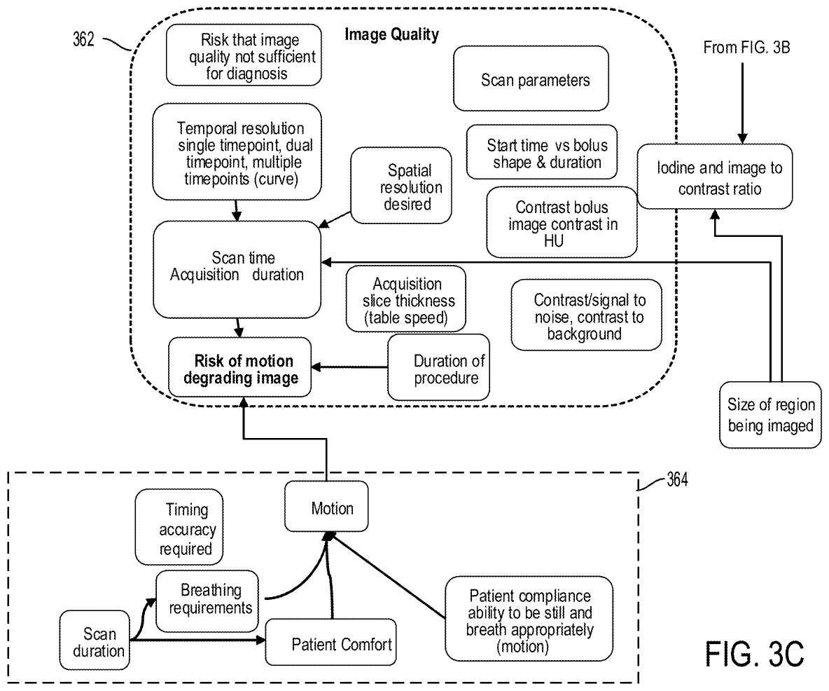

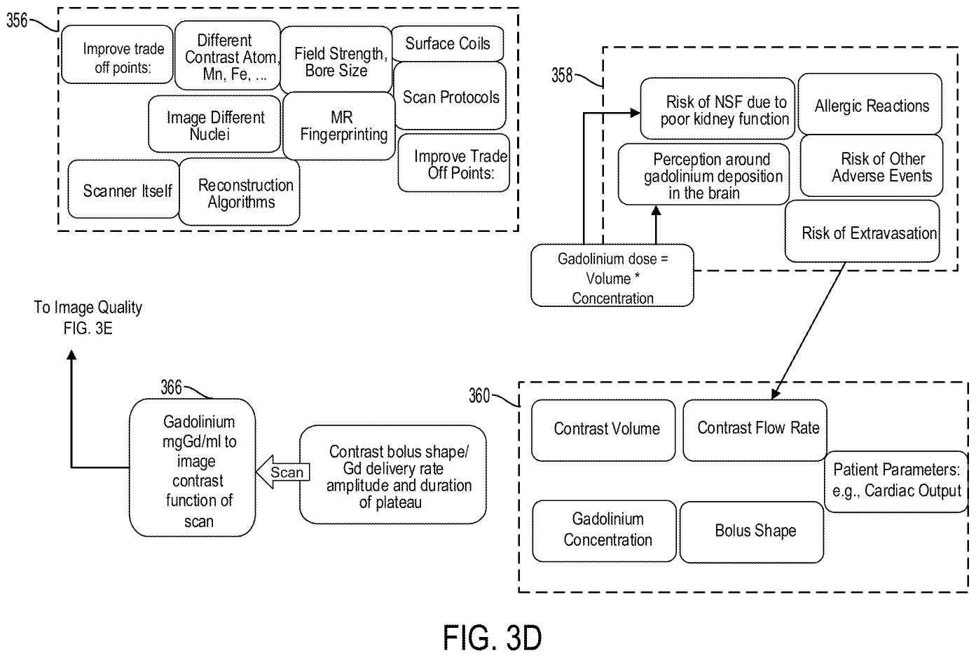

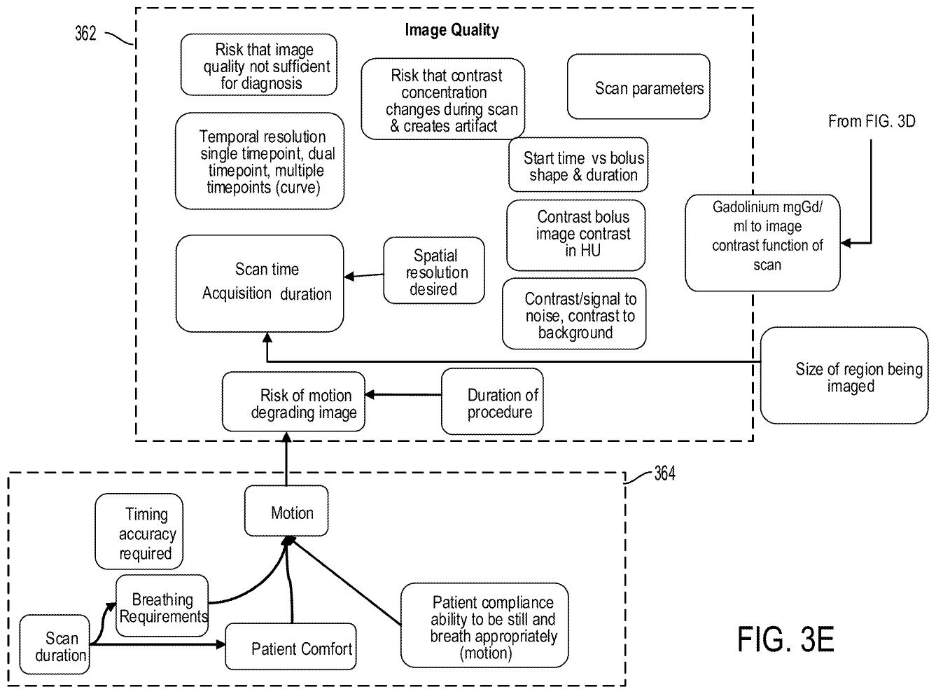

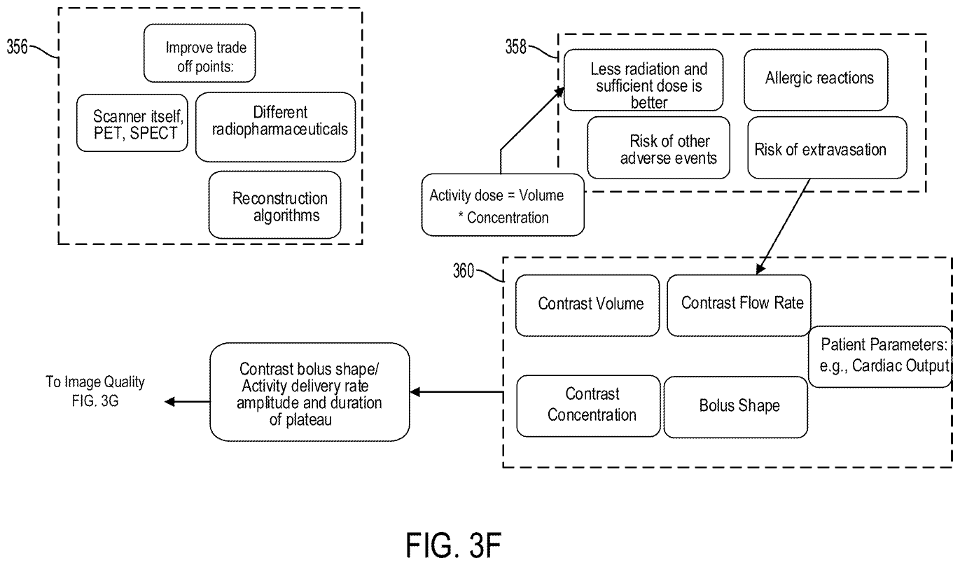

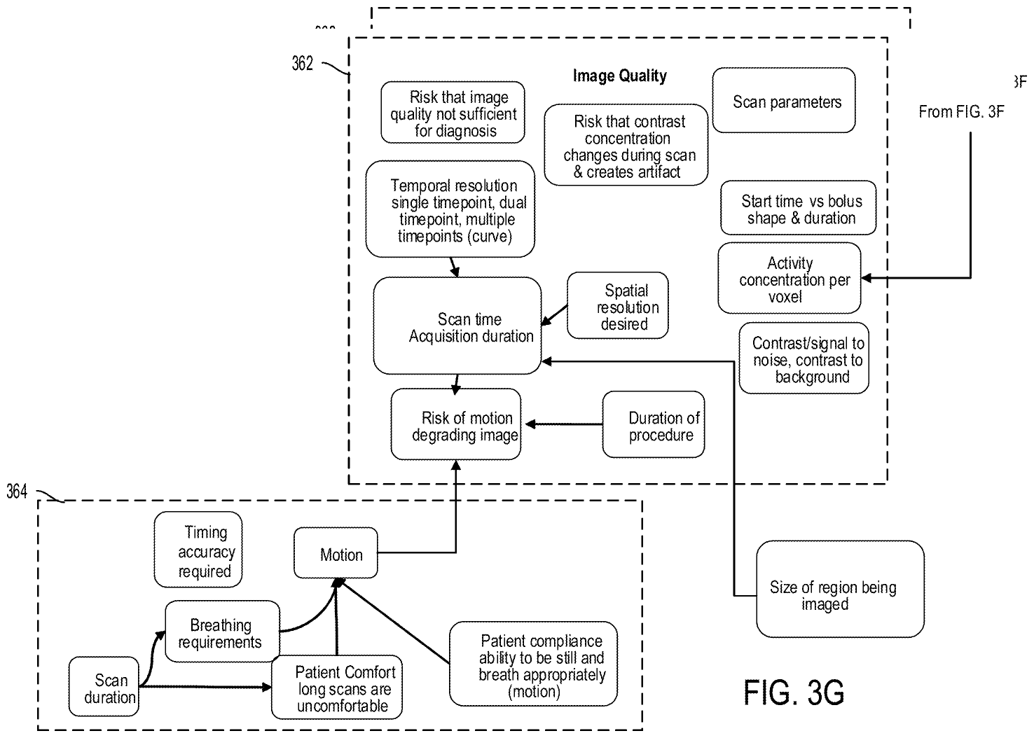

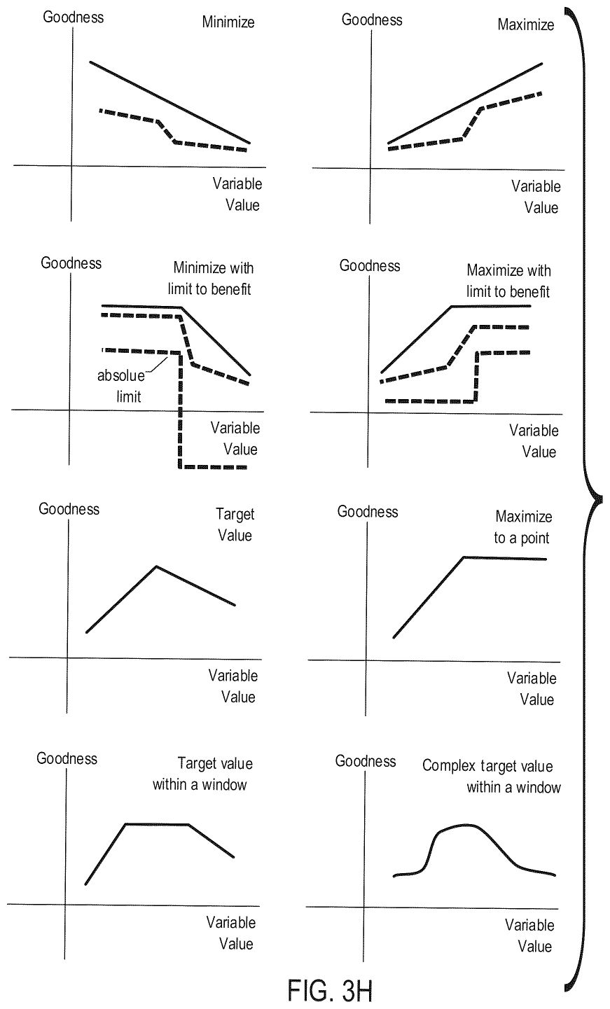

As previously described, the methods and techniques of the present disclosure are provided to modify, customize, and/or adjust baseline or initial protocols generated from current and/or approved models, such as any of the models shown schematically in FIGS. 1A-1D, 2A, and 2B, to create patient-specific protocols for imaging procedures. In particular, the present inventors envision that protocol modification and/or optimization can be accomplished based on consideration of the following major criteria shown in FIG. 3A: depending upon the imaging modality or modalities to be considered, radiation dose or scan time 350, contrast dose 352, and image quality 354. Evaluation of relationships between these three major criteria can be referred to as a trade-off triangle for contrast image acquisition. As imaging equipment improvements occur, improvements such as resolution, sensitivity, speed and other improvements enable a new level overall which may be used to improve one or more of the trade-off parameters. FIGS. 3B-3G are schematic drawings visually depicting relationships between and considerations relevant for the three major criteria (e.g., the trade-off triangle) for different imaging procedures. The methods and techniques described herein take these relationships and considerations into account in order to modify or optimize an initial or baseline protocol to provide the patient-specific protocol.

More specifically, the systems and methods of the present disclosure rely, at least in part, on a hierarchy of optimization to better serve the patient and his or her particular risk factors.

FIGS. 3B-3G represent exemplary descriptions of the optimization hierarchy for CT (FIGS. 3B and 3C), MRI (FIGS. 3D and 3E), and nuclear medicine (FIGS. 3F and 3G), each of which are modalities to which the concepts described herein can be applied. Decisions about the parameters to optimize and the order in which to optimize such parameters, can be made, for example, by person(s) (e.g., referring physician, technician, radiologist, patient) or automatically by the system (e.g., through the use of artificial intelligence (AI), machine learning, or a recommendation engine utilizing patient data). In addition to patient demographic and other data, the optimization decisions can be based, for example, on medical/professional society guidelines, government guidelines, patient preferences, manufacturer/drug package inserts and instructions for use (e.g., for contrast optimization), and other types of trade literature.

As shown in FIG. 3B-3G, parameters for consideration and/or trade-off points (listed in box 356) can include or be related to the scanner itself (e.g., a type of scanner, scanner capabilities, maximum and minimum injection pressures, etc.). Other features for consideration can include reconstruction algorithms, CT imager properties, such as photon counting CT, scanner bed speed, image source, and/or scanner sensitivity and field strength. As shown in FIG. 3D, trade-off points can also include or be related to contrast atoms used (e.g., I, Gd, Mn, Fe, W, etc.), imaging for different nuclei, radiopharmaceutical activity level, surface coil limitations, scan protocols used, field strength/bore size, or MRI fingerprinting.

FIGS. 3B and 3C are schematic drawings showing trade-off considerations for a CT imaging procedure with iodine. As shown in FIGS. 3B and 3C, considerations related to reducing radiation and contrast dose risk (e.g., radiation dose exposure for organs), which are enclosed by box 358, can include possible allergic reactions, iodine volume and/or concentration, risk of kidney harm, and/or risk of an adverse event, such as risk of extravasation. As shown in FIGS. 3B and 3C, the risk of extravasation can be mitigated by adjusting parameters including contrast volume, contrast temperature, contrast flow rate, iodine concentration, bolus shape, or amplitude and duration of the plateau, as shown by box 360. Risk of extravasation can also be mitigated by taking into account patient function information, such as patient cardiac output, or other patient characteristics such as age, vein status/fragility, IV access location, or patient ability to follow instructions and not move during contrast administration.

Considerations for improving image quality (e.g., related to the risk that image quality will not be sufficient for diagnosis), enclosed within box 362, can include adjustments for scan parameters, temporal resolution (e.g., considering s single time-point, dual time-point, and/or a multiple time-point curve), scan time acquisition duration, start time vs. bolus shape/duration, bolus image contrast (in HU), desired spatial resolution, acquisition slice thickness, contrast signal-to-noise or contrast-to-background requirements, duration of the procedure, and/or a size of region being imaged. Considerations for image quality can also include considerations related to risk of motion degrading the image, as shown by box 364. Motion can be caused by, for example, heart motion, breathing, peristalsis, epilepsy, patient inability to control body movement due to anxiety or inability to follow instructions, patient adverse reactions (e.g. feeling of having a hot flash, metallic taste, nausea, increase in heart rate, pain) to the contrast media, and/or any other patient body or limb motion. Motion degradation can be mitigated by changing timing accuracy requirements or breathing requirements or the scan protocol. Motion degradation can also be mitigated by taking action to improve patient compliance and/or reminding the patient of the importance of remaining still and breathing appropriately during image acquisition, or with sedation or antianxiety medication. For example, image timing or duration can be increased or lengthened for patients with good ability to remain still and/or to hold their breath for reasonable periods of time. Image timing or duration can be decreased or shortened for patients that do not remain still even when asked to do so.

FIGS. 3D and 3E shows trade-off features or considerations for an MR imaging procedure with using a gadolinium contrast based agent/GCBA (mgGd/ml), as shown in box 366. Unique considerations (enclosed in box 356) related to radiation dose risk for gadolinium can include gadolinium dose concentration volume considerations (e.g., relevant for risk of gadolinium deposition in brain or risk of nephrogenic systemic fibrosis (NSF) due to poor kidney function), as well as allergic reactions, risk of adverse events, and/or risk of extravasation. As in previous examples, risk of extravasation can be mitigated by, for example, adjustment of contrast bolus shape, Gd delivery rate, amplitude or duration of plateau, injection site, contrast volume, and/or contrast flow rate.

FIGS. 3F and 3G show trade-off features for nuclear medicine (e.g., PET and SPECT) imaging procedures. As shown in FIGS. 3F and 3G, image trade-off considerations (enclosed in box 356) can include requirements for the scanner itself, reconstruction algorithms, and/or different radiopharmaceuticals. Contrast and/or radiation dose risk mitigation features (enclosed in box 358) can include using less radiation or a lower contrast dose, as well as modifications that mitigate allergic reactions, risk of other adverse events, or risk of extravasation. As in previous examples, risk of extravasation can be mitigated or addressed by, for example, adjusting contrast volume, contrast flow rate (e.g., activity delivery rate), contrast concentration, bolus shape, and/or amplitude/duration of the plateau, as shown in box 360. Radiation risk can also be mitigated by taking into account patient function characteristics, such as cardiac output.

Considerations for improving image quality (box 362) can include consideration of whether contrast concentration changes during a scan cause contrast artifacts. Scan parameters, temporal resolution, start time bolus shape/duration, acquisition duration, spatial resolution, activity concentration per voxel, contrast signal-to-noise ratio, contrast background, and/or duration of the procedure can also be adjusted or optimized for improving image quality. As previously described and as shown by box 364, a risk of motion degrading images can also be taken into consideration to improve image quality.

The representations of trade-off triangles in FIGS. 3A-3G show that modifying certain characteristics or parameters can result in an injection protocol that is more beneficial for a particular patient than a standard or baseline protocol. However, in many cases, modifying one variable does not result in a linear improvement of another parameter, such as parameters for image quality. In addition, a change in one parameter may affect multiple other parameters, sometimes in conflicting ways or opposite directions. This idea that changes to a variable result in different and/or non-linear improvements is shown visually in FIG. 3H, which represents a series of graphs or charts illustrating the concept of a “goodness” scale as it relates to different variables. The basic concept illustrated in the graphs of FIG. 3H is that changes in an independent variable value can have different tradeoffs on the “goodness” of a particular dependent or output variable, and that these changes can be, but often are not, linear. For example, increasing contrast dose (an independent variable) may increase the “goodness” of the resulting image quality, but this is likely not a linear relationship. In fact, having too much contrast in CT can lead to streak or star artifacts, hide calcifications in blood vessels, or, in MR, actually decrease the MR signal. Instead, any benefit conferred by an increase in contrast dose to the image quality may reach a maximum. Once the maximum “goodness” for image quality is reached, further increases in contrast dose do not provide any further improvement on image quality. In other examples, as shown by other graphs in FIG. 3H, the “goodness” improvement can be a step function. For example, increasing an independent variable may result in no change in “goodness” until a threshold value is reached.

Once the threshold value is reached, the “goodness” may increase sharply and then level off creating a graph with a step function or waveform. Many other possible “goodness” responses to increasing an independent variable value are shown in the graphs of FIG. 3H.

### Protocol Optimization Method

The present disclosure describes methods and techniques for modifying the initial or baseline protocol based on patient specific and other input data. As previously described, the study protocol can comprise a set of parameter values for a medical injection and imaging procedure related, for example, to injector parameters, parameters related to the contrast media, and/or parameters related to a radiation dose. For example, a generated study protocol can comprise one or more of the following parameters: total contrast volume, maximum flow rate, contrast delivery rate, average flow rate, contrast temperature, contrast viscosity, contrast concentration, IV access location (e.g., position between shoulder and wrist, on a peripheral venous access point on the leg or elsewhere on the body, a central catheter, either arterial or venous), region of scan, potential application to X-ray tube (kVp), maximum current applied to X-ray tube (mAs), scan speed, scan duration, radiation dose, signal/noise (S/N) ratio, contrast/noise (C/N) ratio, or spatial/resolution ratio.

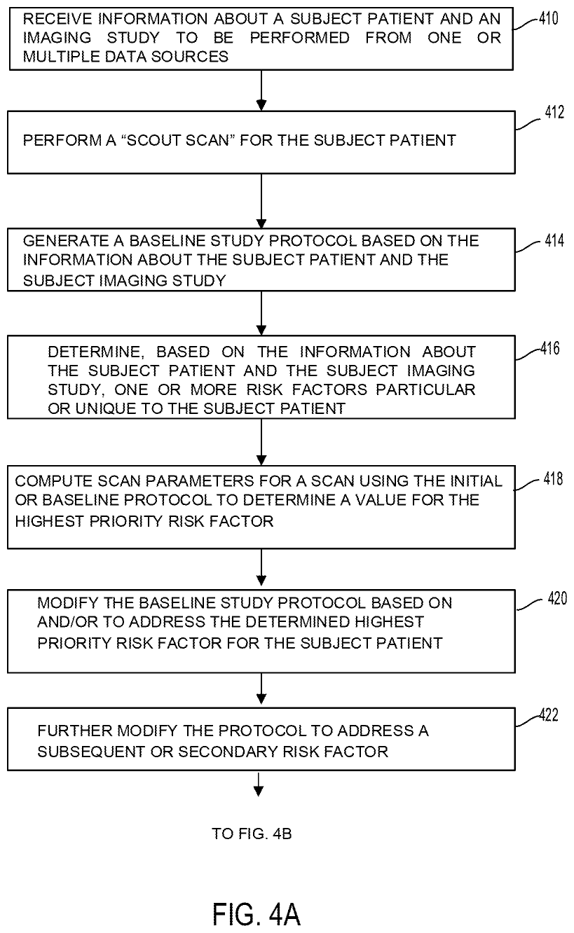

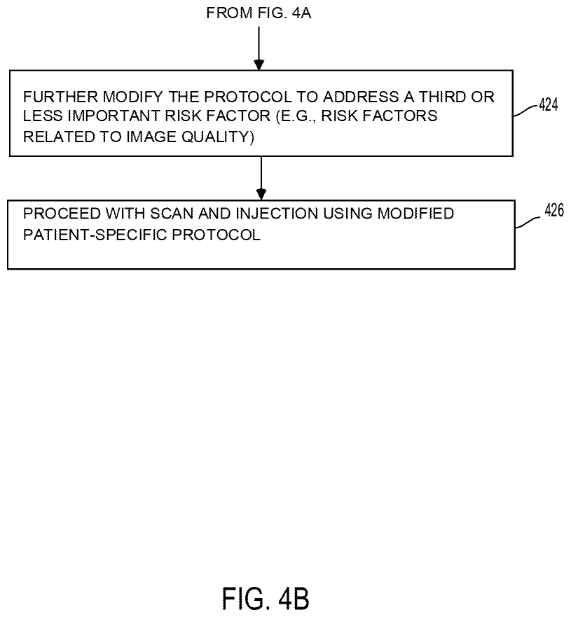

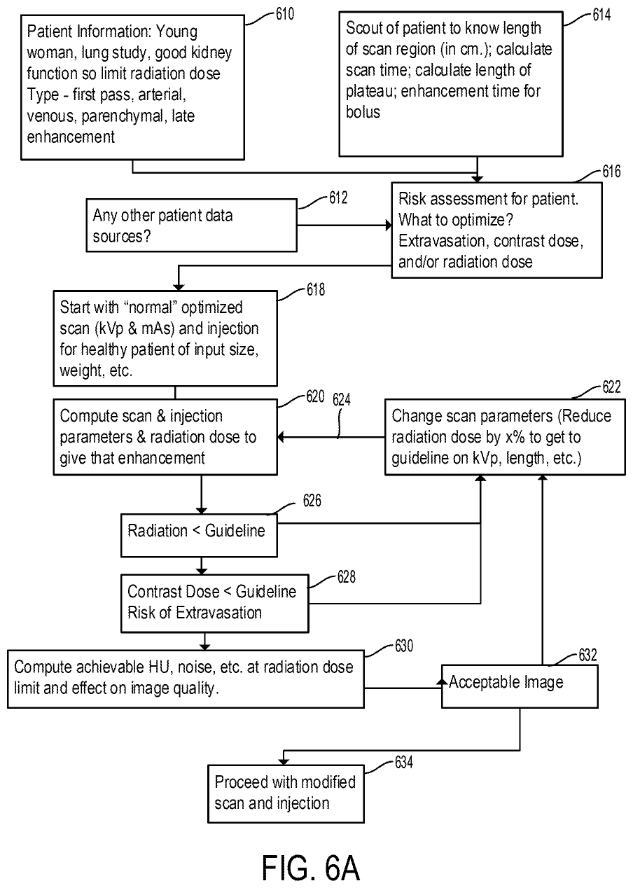

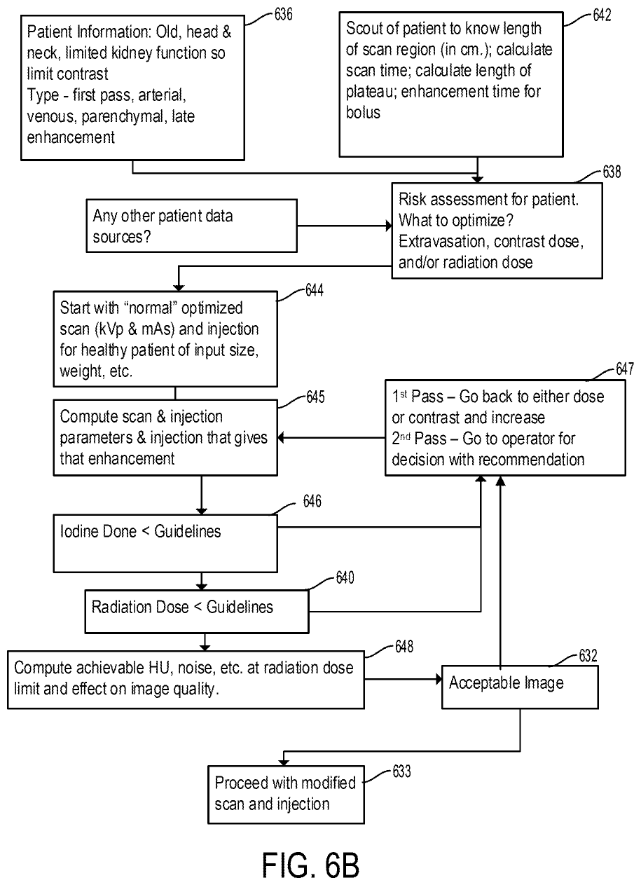

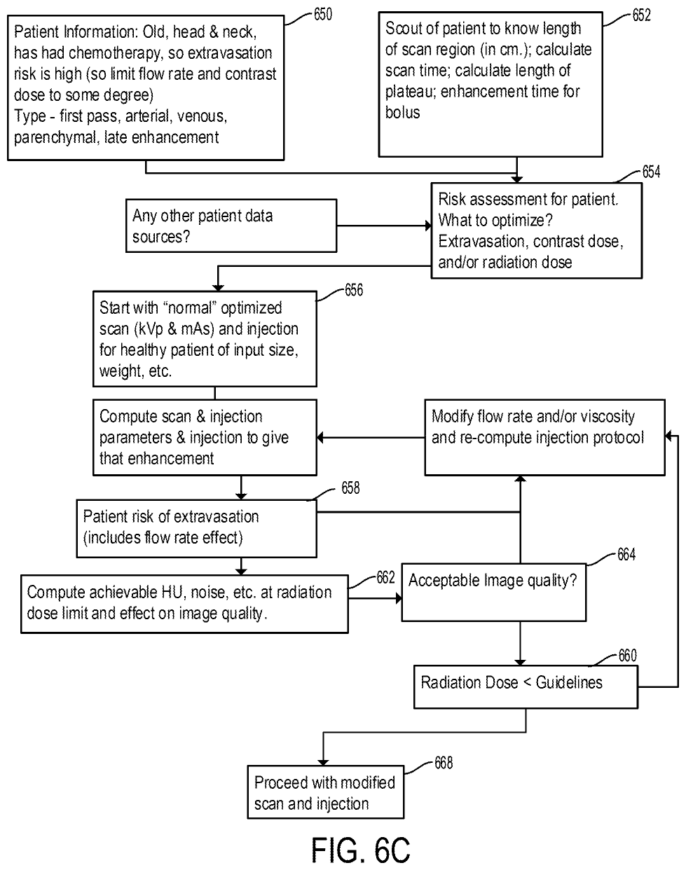

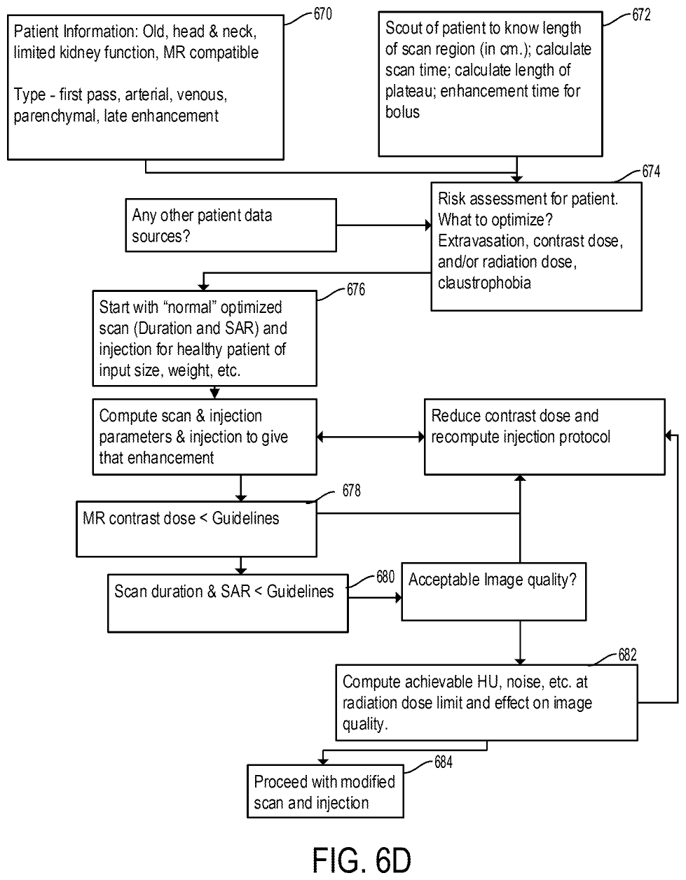

The methods and techniques described herein take into account multiple factors, for example including contrast dose, radiation dose, time required for image acquisition, and image quality considerations and risks, when developing a patient-specific protocol. In particular, the present inventors recognize that some risk factors may be of particular relevance or importance for certain patients, while being of lesser importance for other patients. Therefore, the techniques and methods disclosed herein prioritize some risks based on characteristics unique to the patient and/or procedure being performed. Once risks are identified and prioritized, the initial or baseline study protocol can be modified to account for the unique patient-specific risks. FIGS. 4A and 4B depict a flow chart showing an exemplary method for developing the patient-specific protocol using an iterative process that starts with the initial or baseline protocol for one aspect of the study and modifies the protocol iteratively to arrive at or converge towards the optimized or patient-specific protocol which is then used to select and optimize one or more protocols for other aspects of the imaging study. FIGS. 6A-6D show the method carried out for a specific patient (e.g., a young patient, elderly patient, and/or patient with an underlying disease condition) and the procedure to be performed (e.g., CT, MR, or nuclear medicine) resulting in a generated customized or optimized protocol for the particular patient.

As shown in FIGS. 4A and 4B, the method for developing the patient specific protocol used for a medical imaging study includes, at step 410, receiving information about a subject patient and an imaging study to be performed from one or multiple data sources. The received information can include patient information, such as anthropometric measures for the patient, including height and weight, as well as gender, age, and similar identifying information for the patient. Information about the patient can also include information about patient condition, such as underlying medical conditions. The patent information can be obtained from patient medical records, as well as by asking the patient questions and recording results.

More specifically, in some examples, received patient information can be related to one or more of: height, weight, body mass index, gender, age (e.g., child, adolescent, young adult, adult, elderly), ethnicity, thoracic width, thoracic circumference, medications taken (e.g., beta blockers), underlying medical conditions (e.g., kidney injury, thyroid injury, presence of metal or non-metal implants, history of kidney stones, cancer, expected or known vascular tumors), physical ability (e.g., breath hold duration or ability to remain still), vital signs (e.g., heart rate, resting heart rate, blood pressure, blood glucose, cardiac output, ejection fraction, blood flow velocity, oxygen saturation, serum creatinine level, estimated glomerular filtration rate (eGFR)), pregnant/expecting to become pregnant, genetic predisposition of the subject patient, for example risk of developing cancer, allergies (contraindications or limitations), results of previous imaging exams for the subject patient, or known radiation sensitivity of the subject patient.

Information about the imaging study can include information about the type of imaging procedure being performed. For example, the imaging procedure can comprise computed tomography (CT) imaging, magnetic resonance (MR) imaging, nuclear medicine, PET, SPECT, ultrasound, thermal imaging, infrared (IR) imaging, or combinations thereof. Combined imaging procedures can include, for example, a thermal/IR study; a PET/CT imaging study; a PET/MR imaging study; a SPECT/CT, MR/elastography study; or a study comprising optical and X-ray imaging.

Sources of data for the patient information and/or information about the study can be obtained from numerous sources, including electronic records, databases, and/or information input by a system operator or user. For example, a data source can include an operator's assessment of the patient and medical equipment available for a study to be performed (e.g., assessment of a patient's breath hold ability, ability to follow instructions, need for sedation, flow rate capability, vein IV gauge, etc.). Data sources for patient and procedure information can also include electronic medical records (EMR) systems, hospital data systems (e.g., a hospital information system (HIS), a radiology information system (RIS), a patient procedure tracking system), a radiology analytics system (RAS), a laboratory information system (LIS), a digital pathology system (DPS), a picture archive and communication system (PACS), an electronic health record (EHR) system such as EPIC, a hospital purchase order system comprising an order for a study to be performed for the subject patient (which may include a paper prescription order), a database comprising previous scan results for the patient, or a government guidelines database (e.g., a database with standards for acceptable radiation dose and contrast dose levels for a particular procedure). Patient and procedure information can also be obtained from other databases and records maintained, for example, by a medical facility, medical equipment manufacturer, or government organization. In addition, patient and procedure information can be obtained from the patient, such as through a written or verbal inquiry, a patient chart, wearables, patient screening form, electronic medical records (including those provided by the patient), etc.

In some examples, at step 412, the method also can include performing a scout scan for the subject patient. The scout scan or tomogram can be used to determine study specific information comprising, for example, a length of a scan region, scan time, length of plateau, and/or enhancement time for a bolus. The study specific information can be used along with other patient information for generating initial or baseline protocol values.

At step 414, the method further comprises generating a baseline study protocol based on the information about the subject patient and the subject imaging study. As previously described, the initial or baseline protocol can be determined using a currently available or conventional model, such as the previously described P3TR software model or any of the other models shown in FIGS. 1A-2B. In some examples, a system can include only a single model (e.g., one of the models shown in FIGS. 1A-1D), which is used for all baseline protocol determinations. As previously described, the method may also include allowing a user to select a particular model to use for calculating the initial or baseline protocol from a set of possible models. In other examples, as shown in FIGS. 2A and 2B, outputs or parameters for the initial or baseline protocol can be calculated or determined using multiple models in sequence or according to an iterative process.

At step 416, the method further comprises determining, based on the information about the subject patient and the subject imaging study, one or more risk factors particular or unique to the subject patient. The particular risk factors can include, for example, risks related to radiation dose, contrast dose, risk of extravasation, risk of adverse events, and/or risks that reduce image quality. For example, as described in further detail in FIG. 6A, it may be especially desirable to limit radiation dose for a young patient, especially reducing radiation dose to the breasts in young women. As described in further detail in FIG. 6B, for a patient with limited kidney function, it may be important to limit contrast dose, even if other risk factors need to be increased (e.g., radiation dose may need to be increased to compensate for the decreased intra venous contrast dose). As described in further detail in FIG. 6C, a patient undergoing chemotherapy may be at increased risk of extravasation, meaning that it may be especially important to reduce flow rate and/or contrast fluid viscosity for such patients. In other examples, for patients who cannot remain still or have trouble holding their breath, it may be important to decrease scan duration so that images are not affected by patient movement.

In some examples, determining the risk factors can include prioritizing which risk factors should be evaluated or considered first. For example, as previously described, radiation dose may be of particular importance for young patients. In that case, the protocol may first be optimized to reduce radiation dose. Following optimization to reduce the radiation dose, other models or optimization processes can be applied to the protocol to address issues related to contrast dose and/or image quality. By contrast, for the patient with decreased kidney function, the method can comprise prioritizing minimization of the contrast dose. After contrast dose is minimized, the method can comprise further modifying the protocol to address lower priority risks for the particular patient related to the radiation dose and image quality.

At step 418, once the risk factors are determined and/or a priority for the risk factors is selected, the method further comprises computing scan parameters for a scan using the initial or baseline protocol to determine a value for the highest priority risk factor. For example, when radiation dose is of particular interest, a radiation dose value can be determined and compared to guidelines or acceptable values for the particular patient.

At step 420, the method further comprises modifying the baseline study protocol based on and/or to address the determined highest priority risk factor for the particular or subject patient. In particular, this initial modification can be based on a comparison between the calculated value for the risk factor and acceptable values for the parameter provided in literature. The modification can be an iterative process to optimize parameters of the baseline study protocol to minimize the particular risk factor being considered and generate a modified study protocol that will provide an image of sufficient diagnostic quality.

In some examples, in order to modify or adjust radiation dose, the method can comprise addressing a radiation dose risk factor by reducing the radiation dose by a small amount (e.g., X %) by reducing kVp and/or maximum mAs to ensure that the resulting radiation dose is within guidelines for a particular patient. The radiation dose can then be recalculated using the new scan parameters to determine whether the incremental changes produced a desired result. If a desired result was not achieved, the radiation dose can be decreased by additional successive small changes to the scan parameters until a desired result is achieved. The resulting decrease in radiation increases noise in the image while any decrease in KVp increase sensitivity to contrast, thus enabling less contrast to be used.

Once the desired results are achieved, as shown at step 422, the method next comprises addressing another of the risk factors. For example, the method can comprise determining whether the Hounsfield Units (HU) value and noise in images created according to the modified protocol would be of acceptable quality. Reducing kVp increases the sensitivity to contrast while reducing kVp and mAs generally increases image noise, depending upon many factors. If the images are of acceptable quality, the modified protocol is acceptable for use for an imaging procedure for the patient. If it is determined that the HU value and/or noise prevents obtaining images of acceptable quality, then it may be necessary to adjust parameters, such as increasing the contrast dose to improve images. In other examples, it may be necessary to change the scanner or procedure being performed in order to obtain acceptable images while controlling or reducing radiation dose and contrast dose for a particular patient. In this example, increasing contrast dose is a reasonable first step.

Once the desired results are achieved, as shown at step 424, the method next comprises addressing another of the risk factors. For example, for the young patient, a lower priority risk factor can be related to the contrast dose and/or risk of extravasation. Therefore, the method can compare the contrast dose to the amount allowed or recommended for this patient, a healthy young person in this example. If it is within the allowed amount, no adjustment is made. If it is too high, the method can comprise iteratively decreasing contrast dose by a small amount, such as reducing an iodine dose by a predetermined percent, until the desired result is achieved. Once the desired result is achieved, the protocol can be updated to include the determined or derived contrast dose.

At step 426, once it is confirmed that images of acceptable quality can be obtained using the modified protocol, the method comprises proceeding with the scan and injection in accordance with the modified protocol. If the image quality is not acceptable, the system can repeat any of steps 420, 422, and/or 424. If repeating one or more of these steps does not result in acceptable image quality, the system may inform the user of such a situation, via a user interface, an example of which is shown in FIGS. 5D-1 and 5D-2. The user may accept the system's recommendation or may change one or more of the goals and request that the system repeat the optimization process.

The method for generating the modified protocol for the imaging procedure relies on the ability to prioritize which outputs, trade-offs, and risk factors are most important for a particular patient. Determination of which risk factors should be prioritized and/or are most important to modify can be based on user judgement, user preferences, practice guidelines or recommendations, and/or clinical assessment of patient outcomes for previous imaging procedures. Any or all of these can be included in a look-up table or similar database. Patient information can be used as input for the look-up table or database in order to provide values related to which risk factors should be prioritized or modified.

FIGS. 5A-1, 5A-2, 5B-1 and 5B-2 illustrate an exemplary look-up table including optimizations for CT studies. In FIGS. 5A-1 and 5A-2, information in the columns labeled as “Patient Type/Disease,” “Diagnostic Indication/Region,” and “Situation, Problem(s), Issue” represents examples of independent or non-controllable data which can be used as inputs for obtaining information from the look-up table. The information in the column titled “Patient Specific Procedure Objective” represents examples of patient risk assessments as discussed above and inform the appropriate optimization that should be performed in order to address the independent or non-controllable data related to the patient and/or study. The remaining data illustrates the type of injection/scan parameters that can be optimized in order to achieve each of the Patient Specific Procedure Objectives, along with whether each particular parameter would likely need to be increased or decreased from the baseline protocol in order to address each risk assessment.

More specifically, the table of FIGS. 5A-1, 5A-2, 5B-1 and 5B-2 lists scenarios (shown in FIGS. 5A-1 and 5A-2) where certain parameters of a study protocol should be optimized or modified (e.g., increased or decreased), given greater weight, or prioritized relative to other parameters to obtain improved outcomes. The scenarios listed are for CT diagnostic imaging procedures. However, similar tables for other scans and/or procedures can be prepared by those skilled in the art either based on clinical data (e.g., image quality results from previous scans) or mathematical models for medical imaging procedures.

As shown in FIGS. 5B-1 and 5B-2, parameters (enclosed by box 510) are increased or decreased in the different scenarios. The increase or decrease amounts for each parameter for one of the scenarios are enclosed in box 512 (in FIGS. 5B-1 and 5B-2). Specifically, different parameters can be assigned positive values (e.g., 0.5 or 1) for parameters that should be increased relative to a baseline value in particular scenarios and/or assigned negative values (−0.5 or −1) for parameters that should be decreased relative to a baseline value in particular scenarios. For example, parameters assigned a value of 0.5 can be initially increased by 5%, parameters assigned a value of 1 can be initially increased by 10%, parameters assigned a value of −0.5 can be initially decreased by 5%, and parameters assigned a value of −1 can be initially decreased by 10%. Parameters that are not assigned a value can be unchanged from values for an initial or baseline protocol. As previously described, baseline values can be calculated using currently available algorithms and models (e.g., P3TR Software, CARE Bolus, etc.) shown in FIGS. 1A-2B. Other parameter valuations, weighting schemes, and evaluation methods are also possible.

In some examples, scenarios can be based on patient type/disease status (e.g., male, female, pediatric, adult, geriatric, pregnant or likely to become pregnant, cancer, diabetic, chronic kidney disease, at risk cardiovascular health, prior adverse events, obese, etc.). Scenarios can also take into account a procedure to be performed or region to be scanned. Consideration can also be given to patient specific procedure objectives (e.g., need to minimize extravasation risk, reduce radiation dose, scenarios where diagnosis speed needs to be increased or maximized, etc.).

In one scenario, as shown in the first line of the table of FIGS. 5A-1 and 5A-2, a cardiovascular (CTA) scan is being performed for a pediatric patient with increased risk of extravasation due to small veins. In this scenario, it is desirable to minimize the extravasation risk.

As shown in FIGS. 5B-1 and 5B-2, creators of the table determined that this risk can be mitigated by decreasing contrast parameters including total volume and flow rate (e.g., contrast delivery rate). Also, IV access location is moved along the arm towards the body so that that a larger vein may be accessed. Scan parameters can be modified to increase scan speed and decrease scan duration. Temperature, which inversely reduces contrast agent viscosity, can be increased to body temperature because reducing viscosity via temperature reduces extravasation risk. Also, reducing radiation dose via lower kVp reduces radiation risk, but can result in increased image noise and consequently reduced image quality. Other protocol parameters can be maintained at normal or baseline levels. Protocol modifications for many other scenarios are set forth in the table of FIGS. 5A-1, 5A-2, 5B-1 and 5B-2.

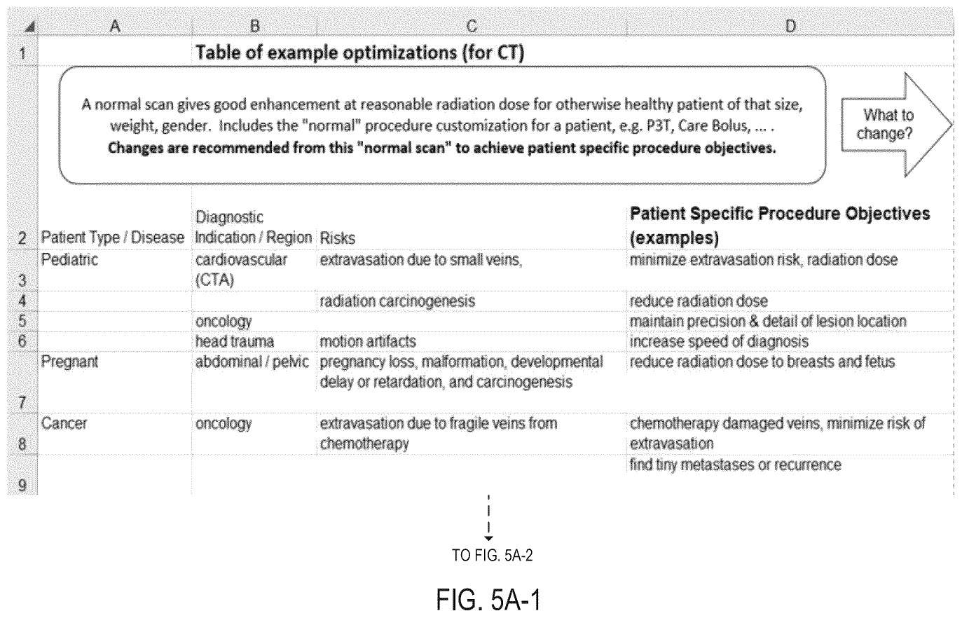

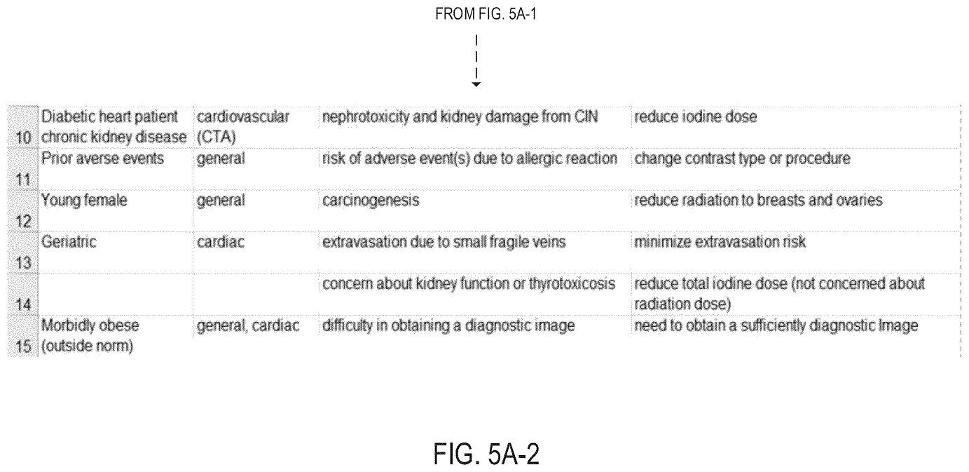

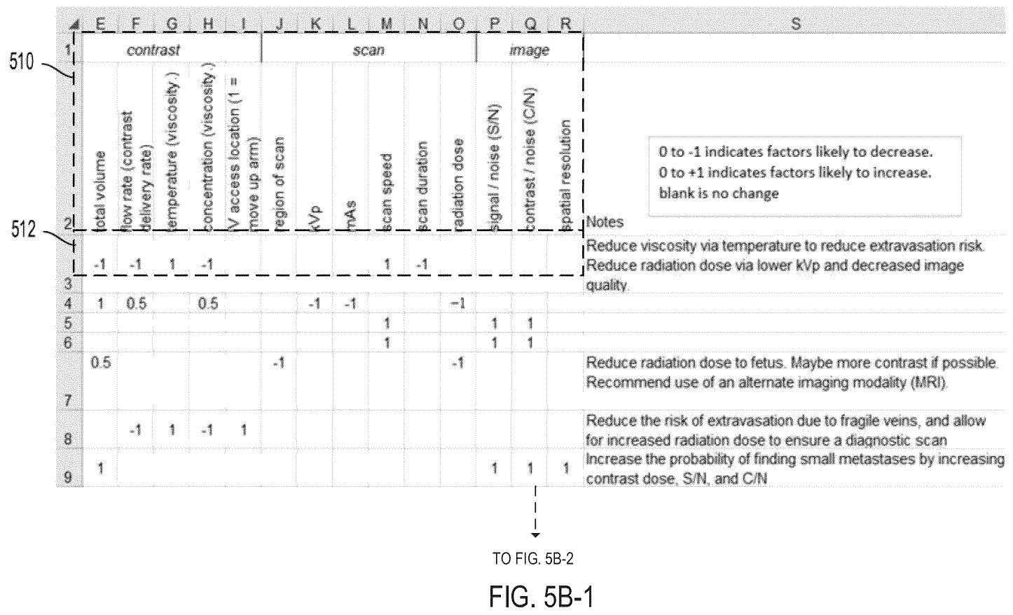

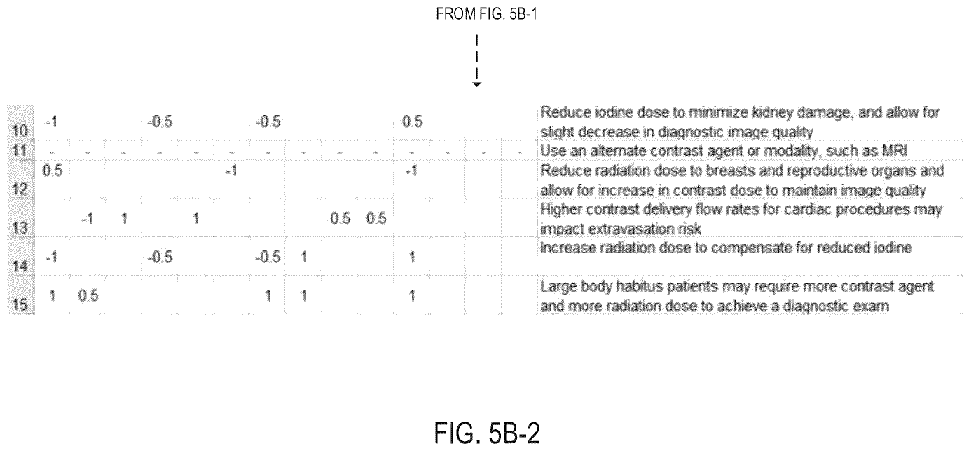

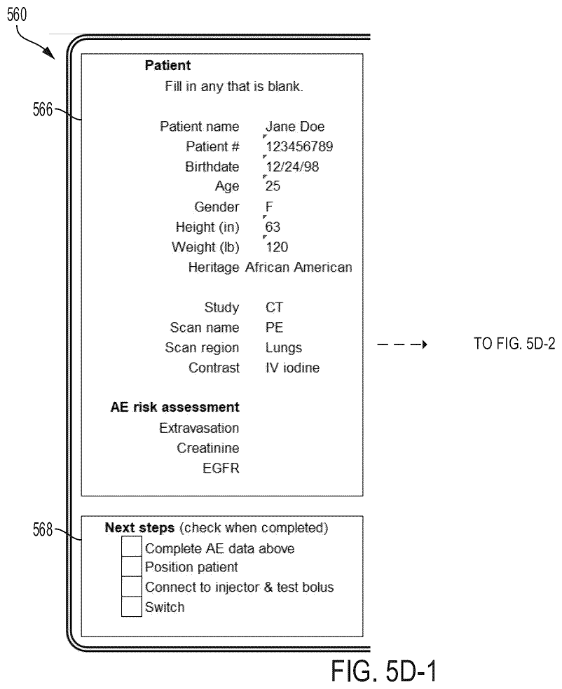

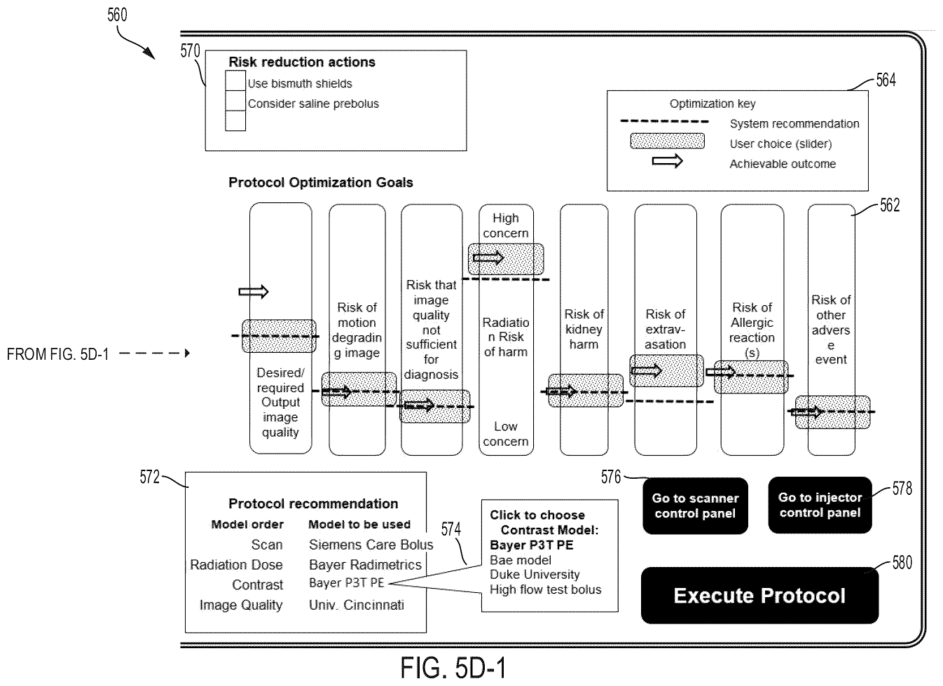

In some examples, a system operator or user may also wish to provide input about which risk factors or patient characteristics should be taken into consideration when optimizing or modifying the initial or baseline protocol. For example, a system operator (e.g., a technologist or physician) may recognize certain features of a patient that should be addressed or taken into consideration based on assessment of the patient. In order to allow the system operator to provide input about such risk factors, the system can include input options allowing the operator to identify risk factors of particular concern with this patient. FIG. 5C is a user interface (e.g., a graphical user interface display screen) providing an exemplary interface that can be provided to the operator for entering inputs related to risk factors of particular concern. As shown in FIG. 5C, the user interface includes user interface elements, which in this example are in the form of multiple bars 550 and slide bar controls 552. However, other user interface elements that allow an input to be provided by a user, such as buttons, virtual knobs or dials, etc. can be used. Each bar is representative of a particular area of risk for a patient that a system operator may wish to address. For example, bars 550 can be related to risk of motion degrading the image, risk of image quality not being sufficient for diagnosis, risk of radiation harm to the patient, risk of kidney harm, risk of extravasation, risk of allergic reactions, and risk of other adverse events. Other risks discussed herein may similarly be represented by slide bars in alternative aspects of this invention. The user interface can also include a bar 554 that provides a desired/required output for image quality. The user interface allows the system operator to move the slide bar controls 552 along the bars 550 to adjust which risk factors are considered to be of primary importance. In one non-limiting embodiment, the user interface is a touch display screen, and the user interface elements can be adjusted by touching the screen.

In some examples, the slide bar controls 552 can be initially positioned based on analysis of patient information and procedure information obtained by the system. The initial position of slide bar controls 552 can be indicated by a dotted line 556 so that the operator can return one or more of the slide bar controls 552 to the initial position and/or recall the initial position in the event a slide bar control 552 moves or is moved, as described below. For example, as previously described, for a young patient, risk of radiation harm can be of primary importance. In that case, the slide bar control 552 on the bar 550 for radiation harm can be near the top of the bar 550, as shown in FIG. 5C. The system operator can move the slide bar controls 552 from their initial position (shown by the dotted lines 556) based on his or her determination of which risk factors are important for the particular patient. For example, the system operator may move the slide bar control 552 for radiation harm downward. In response, the user interface may automatically move other slide bar controls upwards for risk factors, which may increase as radiation dose increases. For example, the slide bar control 552 for the output image quality bar 554 may automatically move upwards, because image quality can improve if the need to mitigate a risk of radiation harm is determined to be lower. In a similar manner, if the system operator moves the slide bar control 552 for the output image quality bar 554 downward, indicating that images of lower quality could be acceptable for a particular imaging study, slide bar controls 552 for bars related to radiation risk or contrast dose risk could move upward indicating that radiation and/or contrast dose could be reduced. The slide bar control 552 position information entered via the user interface of FIG. 5C can be used by the system in order to determine parameter values for optimized or modified protocols using the methods and techniques disclosed herein.

As seen in FIGS. 5D-1 and 5D-2, a user interface 560 may display additional information about the protocol recommendation, including the model order to be used for optimizing various aspects of the protocol, such as contrast, radiation dose, scan, and quality. These models can be selected or changed by the operator. The user interface 560 can also provide a resulting protocol output. The exemplary user interface 560 or control panel (e.g., a graphical user interface display screen) provides for additional features for the overall protocol generation and optimization system. It may be made up of various sections or subpanels. The optimization slide bars referenced in FIG. 5C are in the subpanel 562. A key 564 can explain the function of the various slide bars and slide indicators to the user. Subpanel 566 contains data about the patient collected from various sources, for example electronic medical records (EMR), the hospital information system (HIS), the radiology information system (RIS), patient entered data, or conversation with or observations of the patient which are then entered manually by the operator. The “Next Steps” subpanel 568 indicates (e.g., with boxes) actions which the operator needs to take to indicate that they have been completed.

The Risk Reduction Actions subpanel 570 indicates optional steps which may help to reduce patient risks further. These are actions that are outside the injector or scanner protocols themselves but would be useful for the operator or technologist to perform. Examples include selecting a different catheter gauge or IV location, providing prophylactic pre-treatment with medications for those with a history of or potential for allergic reaction (e.g. Benadryl), providing anti-anxiety medications (e.g. valium) for patients with claustrophobia, or performing pretreatment infusion (e.g. saline or bicarbonate solution) for reducing the occurrence of contrast induced nephropathy for patients that have poor kidney function as discussed in U.S. Pat. No. 9,421,330, entitled “Mitigation Of Contrast-Induced Nephropathy”, the contents of which are incorporated herein by reference.

The protocol recommendation subpanel 572 indicates which models are being used in the protocol optimization process and the order in which the optimization is to happen. When the user clicks on a model, a pop-up 574 opens which allows the user to select one more models to be used for that aspect in the overall imaging modeling and optimization process. Button 576 will take the user to the scanner control panel, and button 578 will take the user to the injector control panel. Button 580 will execute the protocol by loading the appropriate subprotocols into the injector, scanner and any other equipment involved. The scan itself may be started from this user interface, the injector user interface, or the scanner user interface, depending upon the timing between the two, the exact setup of the hardware, and the country regulations on what medical professional has to initiate the use of what medical equipment.

As previously described, FIGS. 6A-6D are flow charts showing specific implementations of the methods for generating protocols of the present disclosure for a specific type of patient and procedure to be performed. For example, in FIG. 6A, existing data about the patient and the study type (e.g., independent or non-controllable data) indicates that the patient is a young woman undergoing a lung study, as shown in box 610. The patient has good kidney function, and the study type may include one or more of first pass, arterial, venous, parenchymal, or late enhancement. As shown by box 612, the system may also query other patient data sources, including prior patient studies. As shown at box 614, the system may also perform the scout scan to determine length of scan region, scan time, length of plateau, enhancement time for the bolus, and any other relevant scan information. Based on the available information from multiple sources, at box 616, the system computes a patient risk assessment to determine what controllable scan/injection parameters (e.g., image acquisition protocol, radiation dose, contrast dose) should be optimized and the order (e.g., priority) of optimization. For this particular patient (young, good kidney function), a patient risk assessment may indicate that the process should first attempt to optimize radiation exposure (radiation dose) and can assign a radiation exposure guideline (e.g., target or threshold value) to be utilized later in the process. The assigned guideline value for this particular patient can be selected from guidelines preloaded into the system, determined by artificial intelligence (AI) and/or machine learning algorithm, and/or entered manually by the operator. The assigned guideline can be based on empirical data reported in, for example, professional literature or contrast package insert materials.

In some examples, the patient risk assessment also identifies other or secondary scan/injection parameters that should be optimized after the radiation dose. For example, contrast dose can be optimized so as to limit the patient's risk of extravasation when the volume or flow rate of contrast meets or exceeds a certain level. To optimize contrast dose, the system assigns a contrast dose guideline (e.g., target, threshold value, or goodness function of FIG. 3H) to be utilized later in the process. The contrast dose guideline can be determined in a similar way as discussed above. An example of such guidance on contrast dose available from trade literature is available in the American College of Radiology Manual on Contrast Media at http://www.acr.org/-/media/ACR/files/clinical-resources/contrast_media.pdf.

As previously described, computation of the optimal study protocol begins, at box 618, with a baseline protocol for a model patient sharing the particular patient demographics (e.g., size, weight, etc.) of the subject patient. Such a baseline protocol can include both scan parameters (e.g., kVp and mAs) and injection parameters (e.g., mL, flow rate), which are capable of achieving a diagnostic image quality in a model patient. A baseline protocol can be determined according to any known method, including using the models shown schematically in FIGS. 1A-2B. As previously described, it is expected that the baseline protocol will provide an adequate enhancement at a reasonable radiation dose for an otherwise healthy patient of the same size, weight, and gender as the subject patient.

At box 620, optimization of the baseline protocol can proceed by analyzing the baseline protocol in view of the patient risk assessment discussed above. In this particular example, this analysis involves determining the radiation dose that will be delivered according to the baseline protocol, based on the scan/injection parameters established for the baseline protocol. The system can then compare this radiation dose value to the radiation exposure guideline value assigned earlier in the process. If the calculated radiation dose that is expected to be delivered using the baseline protocol exceeds the radiation exposure guideline value or the user's selected risk value, the system modifies the scan parameters of the baseline protocol, as shown at box 622, to reduce the radiation dose by a certain amount so as to be at or below the radiation exposure guideline. This can be done, for example, by changing the kVp, scan length, region of interest, direction of radiation (e.g. posterior or anterior vs. full circle) etc. As part of this step, the system can also modify other parameters, including injection parameters, as necessary, to arrive at a potentially acceptable imaging protocol. Consideration may also be made as to whether the modified study protocol can achieve a level of enhancement sufficient to render an acceptable image. Alternatively or in addition, the system may suggest operator actions: centering the patient if that would reduce dose significantly, use of bismuth shielding for breasts. Additional example recommendations may be found in Application of Different methods for Reducing Radiation Dose to Breast during MDCT, PMCID: PMC6280114; PMID: 30568923, available online at:

https://www.ncbi.nlm.nih.gov/pmc/articles/PMC6280114/#:~:text=PMCID%3A%20PMC6280114-
,PMID%3A%2030568923,-Application%20of%20Different.

Once the scan parameters have been adjusted, the system can again determine the radiation dose that will be delivered in this first modified protocol, as shown by arrow 624. After this calculation is complete, the system can again compare the radiation dose to the radiation exposure guideline value, at box 626. If the radiation dose of this first modified protocol remains above the radiation exposure guideline value, the system can again modify the scan parameters to attempt to lower the radiation dose so that it is at or below the radiation exposure guideline value, thereby repeating certain steps described above. If, however, the recalculated radiation dose is at or below the radiation exposure guideline value, the system proceeds to the next step, which involves an assessment of one or more secondary risk factors, which, in this case, is whether the contrast dose is above the guideline or the extravasation risk, as shown by box 628. In this particular example, both contrast volume and flow rate, which related directly to extravasation risk, are handled by a single model, so they are optimized in a single step.

In this particular example, a secondary risk factor assessment involves an analysis of whether the contrast dose to be delivered in the first modified protocol will be at or below the contrast dose guideline and the risk of extravasation at or below its guideline. To make this assessment, the system can compare the contrast dose parameter of the first modified protocol to the contrast dose guideline value assigned above. If the contrast dose parameter of the first modified protocol is above the contrast dose guideline value, or the flow rate high enough that the risk of extravasation will be considered unacceptably high, then another iteration may be done through step 622 with a slightly reduced image enhancement (Hounsfield units) or image quality. The system can then modify the injection parameters of the first modified protocol to lower the contrast dose and risk of extravasation. Once the injection parameters have been adjusted, the system can again calculate the scan and injection parameters necessary to achieve the diagnostic enhancement value and can again determine and assess the radiation dose that will be delivered in this second modified protocol, thereby iterating the above steps with the revised values. On the other hand, if the contrast dose is at or below the contrast dose guideline value, the system can proceed to the next step, which involves computing the image quality (e.g., Hounsfield units, noise, etc.) that is achievable with the updated protocol, as shown by box 630. In this step, the system can confirm that application of the study protocol will allow for an image of sufficient quality for diagnostic purposes. If the system determines that an acceptable image is achievable in box 632, the protocol can be used to perform the study on the subject patient, as shown by box 634. However, if the system determines that an acceptable image is not achievable, the system can perform further iterations by adjusting (usually increasing) the contrast dose and/or radiation dose and again performing the steps described above. Alternatively, the system can suggest a different scanner and/or modality be used. This alternative may be particularly appropriate if multiple iterations have already been performed without arriving at an acceptable optimized or modified patient-specific protocol. Alternatively, the system may inform the user of the situation and the user may proceed with the study because the user believes that it will be sufficient given the patient travel time, cost, and other factors and related tradeoffs that are not yet modeled by this system.

FIG. 6B is a flow chart showing the iterative process for determining the patient specific protocol for another patient. In this example, existing data about the patient and the study type indicates that the patient is an elderly person undergoing a head and neck study, as shown in box 636. The patient has limited kidney function and the study type may include one or more of first pass, arterial, venous, parenchymal, late enhancement. Thus, the study differs from that in FIG. 6A in that the patient is an elderly man (as opposed to a young woman) undergoing a head and neck study (as opposed to a lung study). The two patients have different “importances” or weights on the various risks that occur.

Like with the example in FIG. 6A, the system utilizes available data to compute a patient risk assessment to determine what controllable scan/injection parameters (e.g., radiation dose, contrast dose) should be optimized and the order (e.g., priority) of optimization, as shown in box 638. For this particular patient (elderly, limited kidney function), the patient risk assessment indicates that the process should first attempt to optimize/limit contrast dose and the system assigns a contrast dose guideline (e.g., value) to be utilized later in the process. The contrast dose guideline can be determined in a similar way to FIG. 6A, as discussed above. The guideline can be preloaded into the system, determined by AI, and/or entered manually by the operator, and can be based on empirical data reported in, for example, professional literature or contrast package insert materials. An example of such guidance on contrast dose available from trade literature is available in the American College of Radiology Manual on Contrast Media at http://www.acr.org/-/media/ACR/files/clinical-resources/contrast_media.pdf.

In the example of FIG. 6B, the patient risk assessment also identifies, as a second scan/injection parameter that should be optimized, the radiation dose. The system assigns a radiation dose guideline (e.g., value) to be utilized later in the process (e.g., to be used in the radiation dose compare step 640). The radiation dose guideline can be determined in a similar way as discussed above.

Like with the example in FIG. 6A, a scout scan, at box 642, is performed on the patient to determine the length of the scan region (in cm), the scan time, the length of plateau (useful), and the enhancement time for the bolus. Furthermore, like with the example in FIG. 6A, computation of the optimal study protocol can begin with a baseline protocol for a model patient sharing the particular patient demographics (e.g., size weight, etc.) of the subject patient, as shown by box 644. Such a baseline protocol can include both scan parameters (e.g., kVp and mAs) and injection parameters (e.g., mL, flow rate) that are capable of achieving a diagnostic image quality in a model patient. A baseline protocol can be determined according to any known method, including those mentioned above such as P3TR Software, CARE Bolus, shown in FIGS. 1A-2B. It is expected that the baseline protocol provides an adequate enhancement at a reasonable radiation dose for otherwise healthy patient of the same size, weight, and gender as the subject patient.

The optimization of the baseline protocol can then proceed by analyzing the baseline protocol in view of the patient risk assessment discussed above. In this example, this involves first comparing the contrast dose of the baseline protocol to the contrast dose guideline value assigned for this specific patient earlier in the process, as shown at box 645. If the calculated contrast dose of the baseline protocol exceeds the contrast dose guideline value, the system modifies the injection parameters of the baseline protocol to reduce the contrast dose by a certain amount so as to be at or below the contrast dose guideline. This can be done, for example, by changing the flow rate, iodine concentration, and/or injection duration, as would be appreciated by a person of skill in the art. As part of this step 647, the system can also modify other parameters, including scan parameters, as necessary to arrive at a potentially acceptable study protocol. For example, kVp may be reduced to increase the ratio of Hounsfield unites per (milligrams of iodine per ml) so less contrast may be used, with an increase in the mAs to reduce the image noise so that less Hounsfield units of enhancement are needed, further reducing the contrast dose but at the cost of increasing radiation dose. Consideration may be made into whether the modified study protocol can achieve a level of enhancement sufficient to render an acceptable image.

Once the injection parameters have been adjusted, the system can again compare the contrast dose to the contrast dose guideline value, as shown at box 646. If the contrast dose of this first modified protocol remains above the contrast dose guideline value, the system can again modify the injection parameters and/or contrast dose parameters to attempt to lower the contrast dose so that it is at or below the contrast dose guideline value. If, however, the recalculated contrast dose is at or below the contrast dose guideline value, the system proceeds to the next step, which involves an assessment of the secondary risk factor, which in this case is the radiation dose, as shown by box 640.

In this example, the secondary risk factor assessment involves an analysis of whether the radiation dose to be delivered in the first modified protocol will be at or below the radiation dose guideline. To make this assessment, the system compares the radiation dose parameter of the first modified protocol to the radiation dose guideline value assigned above, as shown by box 640. If the radiation dose parameter of the first modified protocol is above the radiation dose guideline value, the system can then modify the scan parameters of the first modified protocol to lower the radiation dose. Once the scan parameters have been adjusted, the system can again calculate the scan and injection parameters necessary to achieve the diagnostic enhancement value, as shown by box 645, and can again determine and assess the contrast dose that will be delivered in this second modified protocol, thereby iterating the above steps with the revised values. On the other hand, if the radiation dose is at or below the radiation dose guideline value, the system can proceed to the next step, which involves computing the image quality (e.g., Hounsfield units, noise, etc.) that is achievable with the updated protocol, as shown by box 648. In this step, the system can confirm that application of the study protocol will allow for an image of sufficient quality for diagnostic purposes. If the system determines that an acceptable image is achievable, as shown by box 632, the protocol can be used to perform the study on the subject patient, as shown by box 633 in FIG. 6B. However, if the system determines that an acceptable image is not achievable, the system can perform further iterations by adjusting (usually increasing) the contrast dose and/or radiation dose and again performing the steps described above. Alternatively, the system can suggest a different scanner and/or modality be used. This alternative may be particularly appropriate if multiple iterations have already been performed without arriving at an acceptable protocol.

FIG. 6C is a flow chart of a method for developing a study protocol according to the iterative process for another exemplary patient. In this example, the existing data about the patient and the study type indicates that the patient is an elderly person undergoing a head and neck study, as shown in box 650. The patient has previously had intravenous (IV) chemotherapy and the study type may be one or more of first pass, arterial, venous, and parenchymal. Thus, this imaging procedure differ from the examples in FIGS. 6A and 6B in that the patient is an elderly man who has undergone IV chemotherapy. Chemotherapy damages veins and so is a significant risk factor for extravasation. At step 652, a scout scan can also be performed to determine scan information, such as a length of the scan region, scan time, estimation of length of plateau needed, and enhancement time for the bolus.

Like with the examples in FIGS. 6A and 6B, the system utilizes available data for the patient information and/or obtained from the scout scan to compute a patient risk assessment, as shown in box 654, to determine what controllable scan/injection parameters (e.g., radiation dose, contrast dose) should be optimized and the order (e.g., priority) of optimization. For this particular patient (elderly, IV chemotherapy), the patient risk assessment indicates that the patient is at a high risk of extravasation. Therefore, the process first attempts to optimize/limit parameters that coincide with extravasation risk (e.g., contrast dose, contrast viscosity, and flow rate) by assigning an extravasation guideline for this patient (e.g., value which may be based upon contrast dose, viscosity, and flow rate) to be utilized later in the process. The extravasation guideline can be preloaded into the system, determined by artificial intelligence (AI) or machine learning, and/or entered manually by the operator, and can be based on empirical data reported in, for example, professional literature or contrast package insert materials, instructions for use (IFU), and/or drug sheet. The patient risk assessment can also identify, as a second scan/injection parameter that should be optimized, the radiation dose. The system assigns a radiation dose guideline (e.g., value) to be utilized later in the process. The radiation dose guideline can be determined in a similar way as discussed above.

Like with the examples in FIGS. 6A and 6B, computation of the optimal study protocol can begin with a baseline protocol for a model patient sharing the particular patient demographics (e.g., size, weight, etc.) of the subject patient, as shown by box 656. Such a baseline protocol can include both scan parameters (e.g., kVp and mAs) and injection parameters (e.g., mL, flow rate) that are capable of achieving a diagnostic image quality in a model patient. As in previous examples, the baseline protocol can be determined according to any known method, including models, such as P3TR Software, CARE Bolus, etc., shown in FIGS. 1A-2B. It is expected that the generated baseline protocol provides an adequate enhancement at a reasonable radiation dose for an otherwise healthy patient of the same size, weight, and gender as the subject patient.

The optimization of the baseline protocol can then proceed by analyzing the baseline protocol in view of the patient risk assessment discussed above. As shown by box 658, this involves first comparing the extravasation risk of the baseline protocol to the extravasation guideline value assigned earlier in the process. If the extravasation risk of the baseline protocol exceeds the extravasation guideline value, the system modifies the injection parameters of the baseline protocol to reduce the contrast dose, viscosity, and/or flow rate by a certain amount so as to bring the extravasation risk at or below the extravasation guideline. In some examples, the system can also modify other parameters, including scan parameters, as necessary, to arrive at a potentially acceptable study protocol. Consideration may be made into whether the modified study protocol can achieve a level of enhancement sufficient to render an acceptable image.

Once the injection parameters have been adjusted, the system can again compare the extravasation risk to the extravasation guideline value (box 658). If the extravasation risk of this first modified protocol remains unacceptably high, the system can again modify the injection parameters to attempt to lower the extravasation risk so that it is at or below the extravasation guideline value, thereby repeating certain steps described above. If, however, the recalculated extravasation risk is acceptable, the system proceeds to the next steps 660-664, which involve an assessment of the secondary risk factors, which in this case are the image quality and the radiation dose. After some reasonable number of cycles of adjustment, if the risk remains unacceptable, the system may alert the operator to the situation and ask for guidance or intervention. Alternatively, the system may proceed with a greater than desired risk and indicate the achievable outcome on the user interfaces shown in FIGS. 5C, 5D-1 and 5D-2.

In this example, as shown in box 664, the secondary risk factor assessment initially involves an analysis of whether the image quality to be achieved in the first modified protocol will be at or below the image quality guideline. To make this assessment, the system can compare the image quality of the first modified protocol to the image quality guideline value assigned above. If the image quality parameter of the first modified protocol is above the image quality guideline value, the system can then modify the scan parameters of the first modified protocol to increase the image quality. Once the scan parameters have been adjusted, the system can again calculate the scan and injection parameters necessary to achieve the diagnostic enhancement value and can again determine and assess the extravasation risk in this second modified protocol, thereby iterating the above steps with the revised values. On the other hand, if the image quality is at or below the image quality guideline value, the system can proceed to the next step, shown by box 660, which involves computing the radiation dose that is achievable with the updated protocol. In this step, the system can confirm that application of the study protocol will allow for a radiation dose that is sufficiently low. If the system determines that an acceptable radiation dose is achievable, the protocol can be used to perform the study on the subject patient, as shown at box 668. However, if the system determines that an acceptable radiation dose is not achievable, the system can perform further iterations by adjusting (usually increasing) the contrast dose or decreasing image quality and again performing the steps described above. Alternatively, the system can suggest a different scanner and/or modality be used. This alternative may be particularly appropriate if multiple iterations have already been performed without arriving at an acceptable protocol.

FIG. 6D is a flow chart of a method for developing a study protocol according to the iterative process for another exemplary patient who is assumed to be MRI compatible. In this example, as shown in box 670, the existing data about the patient and the study type indicates that the patient is an elderly person undergoing a head and neck study. The patient has limited kidney function. The study type may be one or more of first pass, arterial, venous, parenchymal, or late enhancement MR study. Thus, this imaging procedure differs from the examples in FIGS. 6A-6C in that the study is MRI rather than CT, so the primary trade-off is with duration of scan rather than radiation dose, although SAR (specific absorption rate) and total energy deposited in the patient does need to be limited. There are no known long-term harms from MRI procedures. At step 672, a scout scan can also be performed to determine scan information, such as a length of the scan region, scan duration, length of plateau, and enhancement time for the bolus.

Like with the examples in FIGS. 6A-6C, the system utilizes available data for the patient information and/or obtained from the scout scan to compute a patient risk assessment, as shown in box 674. The risk assessment can be based, for example, on patient characteristics and needs (e.g., age and kidney function). For example, the risk assessment can consider whether risks including extravasation, contrast dose, SAR, patient movement, and/or patient claustrophobia are of primary importance and/or should be addressed for the particular patient. As in previous examples, the risk assessment, at step 674, determines what controllable scan/injection parameters (e.g., SAR, contrast dose, scan time) should be optimized and the order (e.g., priority) of optimization. For this particular patient (elderly, limited kidney function, MRI compatible), the patient risk assessment indicates that risks from the MR contrast dose should be optimized or mitigated, which can be achieved by reducing gadolinium dose by a predetermined amount (e.g., 2%, 5%, or 10%) to get to a guideline value for contrast dose. The guideline value for MR contrast dose can be based, for example, on clinical evidence (e.g., similar procedures performed for similar patients), protocols developed by professional societies, literature, manufacturers (e.g., a contrast package inserts), and/or derived by artificial intelligence. As described below, other risk parameters, which can be optimized after the MR contrast dose, can include scan duration and specific absorption rate (SAR) for the radiation dose. Risk parameters can also relate to image quality (e.g., adjusting signal-to-noise ratio to improve image quality).

Like with the examples in FIGS. 6A-6C, computation of the optimal study protocol can begin with a baseline protocol for a model patient sharing the particular patient demographics (e.g., size, weight, etc.) of the subject patient, as shown by box 676. Such a baseline protocol can include both scan parameters (e.g., SAR and scan duration) and injection parameters (e.g., mL, flow rate) that are capable of achieving a diagnostic image quality in a model patient. As in previous examples, the baseline protocol can be determined according to any known method, including models such as P3TR Software, myExam Companion for MR from Siemens (https://www.siemens-healthineers.com/en-us/magnetic-resonance-imaging/technologies-and-innovations/my-exam-companion), etc., as shown in FIGS. 1A-2B. It is expected that the generated baseline protocol provides an adequate enhancement at a reasonable SAR and scan duration for an otherwise healthy patient of the same size, weight, and gender as the subject patient.

The optimization of the baseline protocol can then proceed by analyzing the baseline protocol in view of the patient risk assessment discussed above. As shown by box 678, this involves first comparing the MR contrast dose value of the baseline protocol to values provided by contrast dose guidelines assigned earlier in the process. If the risk of the baseline protocol exceeds an amount permitted by MR contrast dose guidelines, the system modifies the injection parameters of the baseline protocol to reduce the contrast dose. For example, as previously described, a gadolinium contrast based agent dose can be iteratively reduced by the predetermined amount to come into compliance with guidelines. In some examples, AI tools can be used to reduce the dose by a greater amount, such as 10% to 90%. In other examples, modifying the contrast dose may also include using a higher relaxivity imaging agent, using a different study with a different pulse sequence that provides a different sensitivity to contrast, skipping contrast and using a different study, recommending a different imaging modality, or applying special precautions for a particular patient during pretreatment or post treatment, for example dialysis. In some examples, the system can also modify other parameters, including scan parameters, as necessary, to arrive at a potentially acceptable study protocol. Consideration may be made into whether the modified study protocol can achieve a level of enhancement sufficient to render an acceptable image.

Once the injection parameters have been adjusted, the system can again compare the contrast dose value to a risk value for the contrast dose (box 678). If the contrast dose risk of the first modified protocol remains unacceptably high, the system can again modify the injection parameters to attempt to lower the contrast dose risk so that it is at or below the guideline value, thereby repeating certain steps described above. If, however, the recalculated contrast dose risk is acceptable, the system proceeds to the next step 680, which involves an assessment of the secondary risk factor, which in this case, is the scan duration and SAR.

In this example, as shown in box 680, the secondary risk factor assessment involves an analysis of whether parameters such as scan duration and SAR in the first modified protocol will be at or below the guidelines. To make this assessment, the system can compare the scan duration and SAR parameters of the first modified protocol to the scan duration and SAR guideline values assigned above. If the radiation dose parameter of the first modified protocol is above the radiation dose guideline value, the system can then modify the scan parameters of the first modified protocol to lower the radiation dose. In some examples, radiation dose may also be reduced or mitigated by using a better scanner, applying AI to optimize radiation dose, or utilizing other modalities.

Once the scan parameters have been adjusted, the system can again calculate the scan and injection parameters necessary to achieve the diagnostic enhancement value and can again determine and assess the contrast dose and scan duration and SAR risks in this second modified protocol, thereby iterating the above steps with the revised values. On the other hand, if the scan duration and SAR are at or below the scan duration and SAR guideline values, the system can proceed to the next step, shown by box 682, which involves computing the image quality (e.g., signal and signal to noise ratio, noise, etc.) that is achievable with the updated protocol. In this step, the system can confirm that application of the study protocol will allow for an image of sufficient quality for diagnostic purposes. If the system determines that an acceptable image is achievable, the protocol can be used to perform the study on the subject patient, as shown at box 684. Following performing the study, the patient can be monitored and/or dialysis performed to address concerns about effects of the contrast dose on the patient's kidney function.

If the system determines that an acceptable image is not achievable, the system can perform further iterations by adjusting the scan pulse sequences and again performing the steps described above. Alternatively, the system can suggest a different scanner and/or modality be used. This alternative may be particularly appropriate if multiple iterations have already been performed without arriving at an acceptable protocol.

In each of the examples above, the system develops study protocols through a method in which patient risks are addressed in sequential and, if necessary, an iterative process by identifying and prioritizing the risks and then adjusting the appropriate study parameters so as to reduce the risks to an acceptable threshold. While not explicitly listed in any of the examples above, the system may use the results of a test bolus as discussed above and it may recommend that a test bolus may be done to provide additional information for a better optimization. For example, the system may recommend the performance of a test bolus as described in U.S. Patent Application Publication No. 2022/0133982A1 titled “System And Methods For Delivering A Test Bolus For Medical Imaging”, which is incorporated herein by reference. In addition to addressing the risks to the patient, the image quality is also considered as part of the iterative process in order to ensure that the study protocol can achieve images of sufficient quality that they are useful from a diagnostic perspective. The risk of a poor or non-diagnostic study is a risk to the patient.

### Fluid Injector Systems

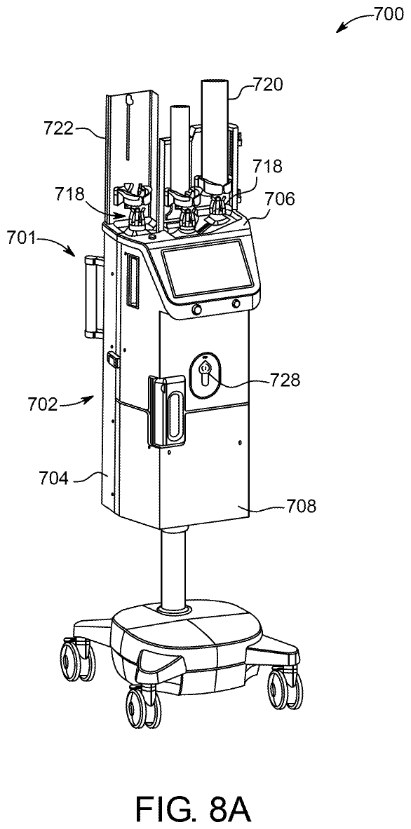

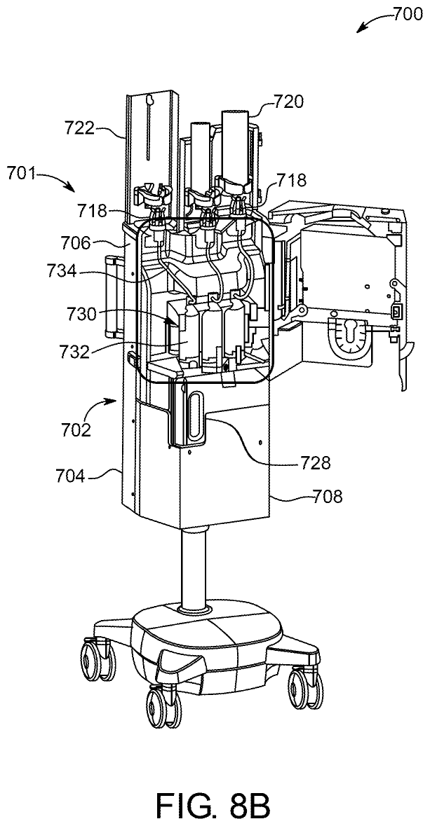

The modified protocols produced using the systems and methods of the present disclosure can be used for obtaining images with fluid injector and image scanner devices and systems known in the art such as is shown in FIGS. 8A-11. For example, once a modified protocol is created and verified, the protocol can be downloaded and stored on device memory of a fluid injector and/or scanner. Once the modified protocol is saved to memory of the injector and/or scanner, the system can proceed to perform their respective functions to obtain images according to the modified protocol. In some examples, modified protocols are generated remote from the fluid injector and scanner devices. For example, computer software and/or a computer system for generating and optimizing the modified protocols can be stored on a cloud server or another remote computer device. The modified protocol can be downloaded and configured to work with the fluid injector devices when ready for use. In other examples, software for generating the initial or baseline protocol and the modified protocol can be integrated with and/or stored on memory of the fluid injector or scanner. When the fluid injector is activated, the software can be configured to process available data to generate the modified protocol and, once the final modified protocol is created, adjust operating parameters of the fluid injector and scanner in accordance with the modified protocol.

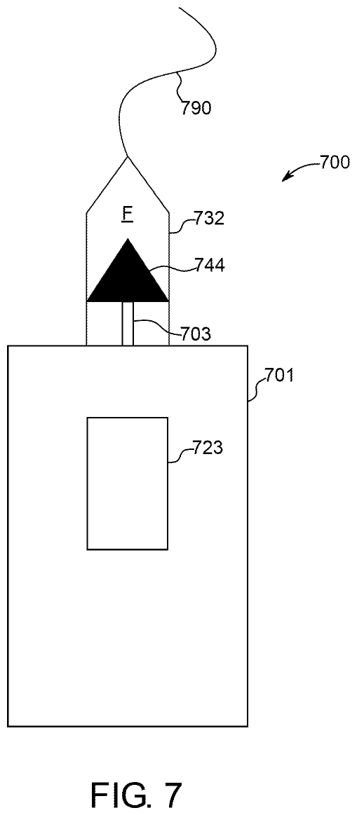

Features of an exemplary fluid injector and fluid injector system which can be adapted for use with the methods and techniques for protocol generation described herein are shown in FIGS. 7-11. Furthermore, although the following description is primarily for a CT Injection System, it is also recognized that the techniques and methods disclosed herein can be applied to a variety of other injection systems. Examples of such injection systems include the MEDRAD® Stellant and MEDRAD® Stellant FLEX CT Injection Systems, the MEDRAD MRXperion MR Injection System, the MEDRAD® Mark 7 Arterion Injection System and the MEDRAD® Centargo CT Injection System offered by Bayer HealthCare LLC.

FIG. 7 shows a schematic drawing of an exemplary fluid injector system 700 having at least one reservoir, such as a syringe 732, in fluid connection with a fluid path set. The fluid path set can be the single-use disposable set (SUDS) 790. The at least one syringe 732 can be configured to be filled with at least one fluid F, such as contrast media, saline solution, or any desired medical fluid. In particular, the contrast media can be a dose of contrast selected according to the modified protocol for a particular patient, as previously described. The at least one fluid F from the at least one syringe 732 can be delivered to a patient using the SUDS 790. The at least one syringe 732 may be pre-filled or it may have the ability to be filled with the at least one fluid F. The at least one syringe 732 may be, for example, a rolling diaphragm syringe, bottle, or collapsible bag.

The system 700 further includes a fluid injector 701, such as an automated or powered fluid injector, which is configured to deliver the fluid F from the syringe 732 to a patient. For example, the injector 701 may be configured to drive a plunger 744 of the syringe 732 with a drive member 703, such as a piston, to deliver the fluid F from the syringe 732 via the fluid path set 790 at an injection speed in compliance with the modified protocol generated for the patient. The at least one drive member 703 may be reciprocally operable to selectively fill the at least one syringe 732 or deliver fluid F from the at least one syringe 732. In some examples or aspects, the injector 701 may be configured to releasably receive the syringe 732. The injector 701 may be a multi-syringe injector, wherein several syringes may be oriented side-by-side or in another spatial relationship and are separately actuated by respective pistons associated with the injector 701.

Fluid flow from the at least one syringe 732 may be regulated by a fluid control module or controller 723 that is configured to operate various valves, stopcocks, and flow regulating structures to regulate the delivery of the at least one fluid F to the patient based on injection parameters (e.g., injection flow rate, duration, and total injection volume) of the modified protocol being implemented.

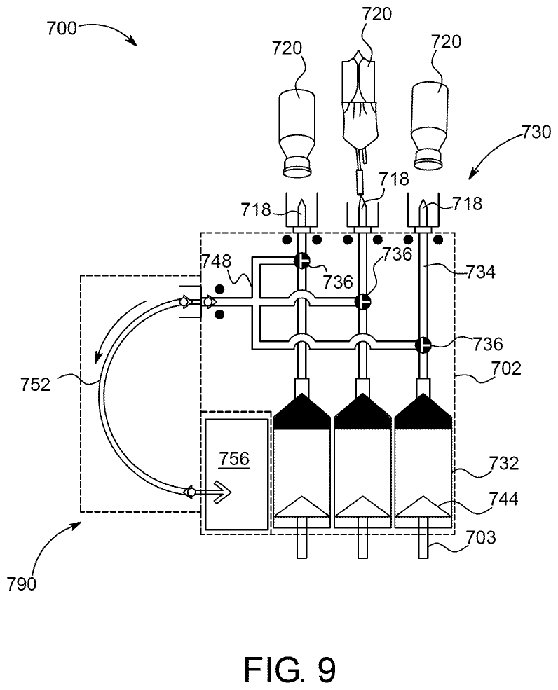

Another exemplary fluid injector system 700 which can be adapted to perform an injection and be used in connection with a scanner to obtain images in accordance with a modified protocol, as described herein, is shown in FIGS. 8A and 8B. Unlike in the previous example, the syringes or fluid reservoirs 732 are positioned in the injector 701, as shown in FIG. 8B. The exemplary fluid injector system 700 includes the powered fluid injector 701 connected to a fluid delivery set intended to be associated with the injector device 701 to deliver fluids from one or more single-dose or multi-dose containers and fluid path sets under pressure into a patient. The fluid injector 701 includes an injector housing 702 with opposed lateral sides 704, a distal or upper end 706, and a proximal or lower end 708. The housing 702 encloses the various mechanical drive components, electrical and power components necessary to drive the mechanical drive components, and control components, such as electronic memory and electronic control devices (hereinafter electronic control device(s)), used to control operation of reciprocally movable drive members, such as drive members 703 (shown in FIGS. 7 and 9) associated with the fluid injector system 700. Such drive members 703 may be reciprocally operable via electro-mechanical drive components, such as a ball screw shaft driven by a motor, a voice coil actuator, a rack-and-pinion gear drive, a linear motor, and the like.

The fluid injector system 700 can further include at least one bulk fluid connector 718 for connection with at least one bulk fluid source 720. Alternatively, the fluid source could be a single dose vial, rather than a bulk source. In some examples or aspects, a plurality of bulk fluid connectors 718 may be provided. For example, as shown in FIGS. 8A and 8B, three bulk fluid connectors 718 may be provided in a side-by-side or other arrangement. In some examples, the at least one bulk fluid connector 718 may be a spike configured for removably connecting to the at least one bulk fluid source 720, such as a vial, bottle, or a bag. The at least one bulk fluid connector 718 may have a reusable or non-reusable interface with each new bulk fluid source 720. The at least one bulk fluid source 720 may be configured for receiving a medical fluid, such as saline, an imaging contrast solution, or other medical fluid, for delivery to the fluid injector system 700. The housing 702 may have at least one support member 722 for supporting the at least one bulk fluid source 720 once it is connected to the fluid injector system 700.

With reference to FIG. 8B, the fluid injector system 700 may further include the disposable components for conveying the fluid to the patient. For example, the fluid injector system 700 can include the multi-use disposable set or MUDS 730 positioned inside the housing 702. Examples and features of the MUDS are described in detail in International Application No. WO 2016/112163 titled “Multiple Fluid Delivery System with Multi-Use Disposable Set and Features Thereof,” the disclosure of which is incorporated herein by reference.

In some examples, the MUDS 730 can include one or more syringes or pumps 732 connected to and/or in fluid communication with the bulk fluid source(s) 720 through a MUDS fluid path 734. In some examples, the number of syringes or fluid reservoirs 732 may correspond to the number of bulk fluid sources 720. For example, the MUDS 730 can include three syringes/fluid reservoirs 732 in a side-by-side arrangement such that each syringe/reservoir 732 is fluidly connectable to one or more of the bulk fluid sources 720.

The MUDS 730 can be removably connected within the housing 702 of the fluid injector system 700 for delivering one or more fluids from the one or more bulk fluid sources 720 to the patient. As described in further detail herein, the fluid injector system 700 can also include sensors for identifying when the MUDS 730 is connected to and/or removed from the fluid injector system 700.

The MUDS 730 may be configured for delivering fluid to a fluid path set, such as the SUDS 790, as described herein. In order to establish fluid communication between the MUDS 730 and the SUDS 790, the fluid injector system 700 can further include at least one slot or connection port 728 for releasably connecting the single-use connector or disposable set (e.g., the SUDS 790) to the MUDS 730.

The SUDS 790 can include, for example, a connector configured to be received by the connection port 728. The connection port 728 can include sensors for identifying when the SUDS 790 is connected to the port 728. The SUDS 790 can further include a patient line for conveying fluid from the MUDS 730 to the patient. Exemplary SUDS 790 are described and shown in International Appl. Pub. No. WO 2015/106107 titled “Single-Use Disposable Set Connector,” which is incorporated herein by reference.

FIG. 9 shows a schematic drawing of components of the MUDS 730 enclosed within a housing 702 of the fluid injector system 700. As shown in FIG. 9, a syringe plunger 744 is disposed within each syringe 732 and is reciprocally movable within the syringe 732 due to movement of a drive member 703 associated with the fluid injector system 700. Each syringe 732 is in fluid communication with a valve 736 which provides fluid communication with a manifold 748 and bulk fluid connector 718. The manifold 748 may also provide support for the syringes/fluid reservoirs 732 so that the syringes/reservoirs 732 can be handled as a single, unitary structure. The manifold 748 may be in fluid communication via the valves 736 and/or the syringes/reservoirs 732 with a first end of the MUDS fluid path 734 that connects each syringe/reservoir 732 to the corresponding bulk fluid source 720. The opposing second end of the MUDS fluid path 734 may be connected to the respective bulk fluid connector 718 that is configured for fluidly connecting with the bulk fluid source 720.

In some examples, when not connected to the patient's catheter (e.g., prior to fluid injection), a patient line 752 of the SUDS 790 can be connected to a waste reservoir 756 on the fluid injector system 700. The waste reservoir 756 is desirably separate from the syringes/fluid reservoirs 732 to prevent contamination. In some examples, the waste reservoir 756 is configured to receive waste fluid expelled from the syringes/reservoirs 732 during, for example, a priming operation. The waste reservoir 756 may be removable from the housing 702 in order to dispose of the contents of the waste reservoir 756. In other examples, the waste reservoir 756 may have a draining port (not shown) for emptying the contents of the waste reservoir 756 without removing the waste reservoir 756 from the housing 702. In some examples, the waste reservoir 756 is provided as a separate component from the MUDS 730.

Exemplary fluid injection systems also include those that are disclosed in the following patents: U.S. Pat. Nos. 6,643,537; 7,094,216; 7,556,619; 8,337,456; 8,147,464; and 8,540,698, the disclosures of each of which are incorporated herein by reference.

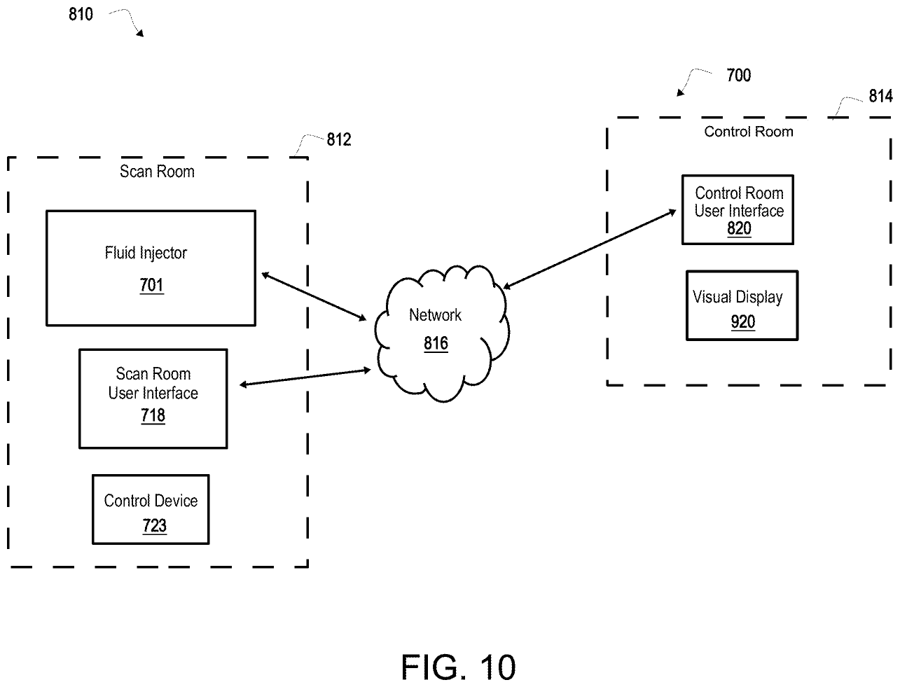

With reference to FIG. 10, the fluid injector system 700 can be configured to be used in an environment 810 including a scan room 812 and a control room 814. For example, the fluid injector system 700 can be a bifurcated system in which some functions, processing, and control operations are performed by devices located in the scan room 812 and other functions, processes, and control operations are performed by devices, processors, and displays located in the control room 814. In particular, processes related to determining the modified protocol can be performed on controllers and other processors in the control room 814. Once the modified protocol is determined, the modified protocol can be provided to computing devices in the scan room 812, which can be configured to control the fluid injector and image scanner to obtain images in accordance with the modified protocol. In some examples, the devices in the different rooms 812, 814 can communicate via a wired or wireless computer network 816.

As shown in FIG. 10, the fluid injector 701 and associated control device 723 are located in the scan room 812. The control device 723 can be configured to provide a scan room user interface 718 for controlling the injector 701 from the scan room 812. The scan room user interface 718 can be located on the fluid injector 701 and, for example, can include a display 920, such as a touch screen display and associated buttons, on the injector housing 702 for operating the injector 701. A user, such as a medical technologist, can review injection parameters, such as the parameters of the modified protocol, and perform other actions to prepare the injection. The scan room user interface 718 can also provide feedback to the user, such as feedback informing the user when the injector 701 is primed and ready to begin performing an injection protocol.

In some examples, the control room 814 can be a shielded control room outside the scan room 812. From the control room 814, the user, such as the medical technologist, can monitor the fluid injector system 700 during an injection protocol in a safe and convenient location. The control room 814 can include a computer device, such as a computer terminal, including one or more controllers or processors for controlling operations of the fluid injector system 700 and fluid injector 701 from the control room 814. The computer devices or terminal in the control room 814 can provide a control room user interface 820 that allows the user to input instructions to the system 700 and injector 701 and to receive feedback from the fluid injector system 700. Feedback can include information about a progress of an injection protocol and, for example, a confirmation when an injection protocol is complete. The feedback can be provided on the visual display in the control room 814.

Some processes related to the generation of the baseline and/or modified protocol studies can be performed by the computer device or terminal in the control room 814. For example, the computer terminal in the control room 814 may be configured to receive information about the patient and/or results of the scout scan. The computer terminal can be configured to generate the baseline protocol or the modified protocol based on the received information. Once a protocol is generated, the computer terminal can be configured to cause the fluid injector 701 and other medical devices to perform the protocol.

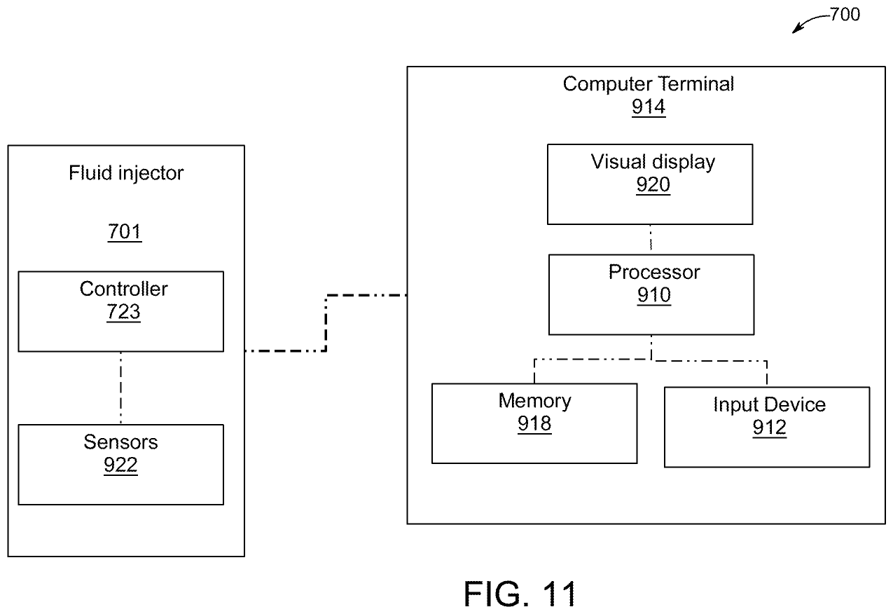

With reference to FIG. 11, electrical components of the fluid injector system 700 are shown and described in detail. As discussed previously, some electrical components and processing circuitry can be located in the scan room 812. Other electrical components and processing circuitry of the fluid injector system 700 can be located in or can be accessed from the control room 814.

In some examples, the fluid injector system 700 includes at least one control device, such as a computer processor 910, which can be configured to generate or modify a protocol using the methods described herein. In some examples, the control device or processor 910 is a processor of the controller 723 (shown in FIG. 7) of the fluid injector 701 located in the scan room 812. In other examples, as shown in FIG. 11, the control device or processor 910 can be a separate processing component remote from and in communication with the controller 723 of the injector 701. For example, the processor 910 could be a component of a control terminal 914 located in the control room 814 or at another location remote from the injector 701. The computer terminal 914 can be configured to control the fluid injector system 700. In other examples, the processor 910 could be a component of a general computer device, such as a computer tablet, smart phone, or laptop computer in communication with and configured to receive information from the controller 723 of the injector 701.

As shown in FIG. 11, the control terminal 914 can include the processor 910, the visual display 920, system memory 918 for storing information about the injector protocols, and one or more input devices 912 for entering information about the injection protocols being performed. The at least one processor 910 of the fluid injector system 700 is configured to receive or determine information about an injection procedure performed by the fluid injector system 700. For example, the information can include a time that an injection is performed.

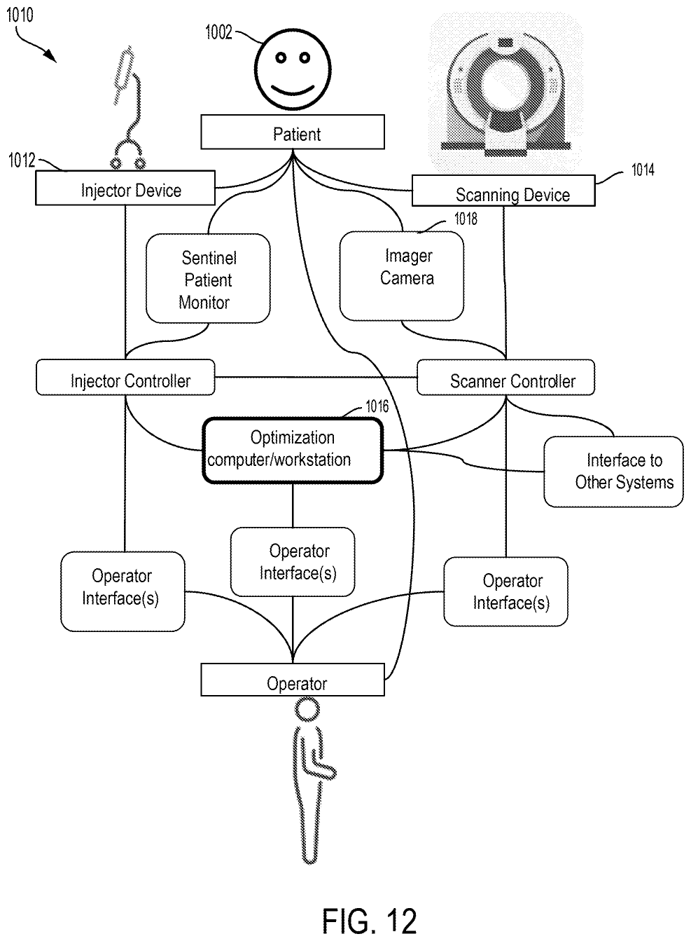

FIG. 12 is an example image creation system 1010 according to certain non-limiting embodiments of the present disclosure. The system 1010 contains an injector device 1012, a scanning or imaging device 1014, and an optimization computer or engine 1016. The injector device 1012 in this example further comprises a patient monitoring device, for example a patient monitoring system as described in International Appl. Pub. No. WO 2021/222771A1 titled “System, Device, And Method for Safeguarding Wellbeing of Patients for Fluid Injection,” which is incorporated herein by reference. Such a patient monitoring device may collect information about a patient 1002 before and/or during the imaging procedure. That information may be used by the optimizing computer or engine 1016 in the optimization process of the present disclosure including the suggestion of risk reduction actions. The imaging device 1014 in this example of FIG. 12 further includes an imager camera 1018, which monitors the patient 1002. The imaging device 1014 may further include an ECG monitor (not shown) or other sensors (not shown) for monitoring the patient 1002. Similarly, these sensors may collect information before and/or during the imaging procedure. Such information, for example heart rate, movement propensity, or respiration rate, is most conveniently and effectively collected by such sensor(s) before the procedure and may be used by the optimization computer 1016 in the optimization process of the present disclosure to adjust risks and/or protocols including the suggestion of risk reduction actions. For example, a patient 1002 who moves significantly before the study has a higher risk of having motion result in poor image quality. The optimizing computer 1016 functions may be performed by a suitable computer as part of the injector 1012, the imaging device 1014, or on an external or cloud computer.

Each of the models described herein encompass certain aspects of the overall imaging protocol. It is anticipated that new models will be developed which may cover more or fewer aspects than those described herein. For example CT scan design, radiation dose estimation, and image quality estimation may be encompassed as a single model or a family of connected or interrelated models. There may also be specific models for specific studies as is the case for the P3TR protocols of Bayer Healthcare LLC or the FAST CARE technology of Siemens as listed at http://www.siemens-healthineers.com/it/computed-tomography/technologies-innovations/fast-care. These more encompassing models may be used as part of the present disclosure with the potential benefits of faster protocol development, more customization and precision of the protocol to the patient, and reduced overall risk to the patient.

Although the disclosure has been described in detail for the purpose of illustration based on what are currently considered to be the most practical and preferred aspects, it is to be understood that such detail is solely for that purpose and that the disclosure is not limited to the disclosed aspects, but, on the contrary, is intended to cover modifications and equivalent arrangements. For example, it is to be understood that the present disclosure contemplates that, to the extent possible, one or more features of any aspect can be combined with one or more features of any other aspect.

1. A system for generating protocols that can be used for medical imaging studies, comprising:
one or more processors;
non-transitory, computer readable media comprising instructions stored therein, wherein the instructions, when executed by the one or more processors, will cause the system to:
receive, from one or more data sources, information about a subject patient and information about a subject imaging study;
determine, based on the information about the subject patient and the information about the subject imaging study, one or more risk factors particular to the subject patient;
select two or more models, wherein the two or more models comprise at least one model of each of at least two aspects of the subject imaging study; and
apply the two or more models to generate a baseline study protocol for the subject imaging study, wherein the baseline study protocol is based upon at least the information about the subject patient and the one or more risk factors, and wherein the baseline study protocol comprises parameters of the at least two aspects of the subject imaging study.

2. The system according to claim 1, wherein the baseline study protocol comprises a contrast injection protocol comprising at least a total contrast dose and a maximum flow rate, and an image acquisition protocol comprising at least scan parameters, scan duration, a timing parameter for coordination with a contrast injection, and one or more image reconstruction algorithms.

3. The system according to claim 1, wherein the two or more models comprise a plurality of models of one aspect of the imaging procedure, wherein the two or more models are configured to operate in parallel to transform a same or similar inputs.

4. The system according to claim 1, wherein the two or more models are applied in a sequence, wherein the sequence is determined based upon at least one or more patient characteristics and a desired optimization of the one or more risk factors.

5. The system according to claim 4, wherein the system is configured to allow a user to accept or change the sequence based on the user's knowledge or preference.

6. The system according to claim 1, wherein the instructions, when executed by the one or more processors, will additionally cause the system to:
perform one or more iteration cycles through at least one of the two or more models to optimize one or more of the parameters of the baseline study protocol.

7. The system according to claim 6, wherein the instructions, when executed by the one or more processors, will additionally cause the system to:
present to a user in a selectable format an outcome of one or more of the iteration cycles if none of the iteration cycles provides an optimized outcome.

8. The system according to claim 1, further comprising a user interface, wherein expected parameters of the baseline study protocol are displayed for user confirmation or further adjustment.

9. The system of claim 8, wherein the user interface provides one or more selectable user interface elements which allows an operator to adjust the one or more risk factors particular to the subject patient.

10. The system of claim 9, wherein at least one of the one or more selectable user interface elements is in the form of a slider bar that is adjustable by a user.

11. The system of claim 10, wherein the user interface is a graphical user interface display screen, and the one or more user interface elements can be adjusted by a touch of the user on the graphical user interface display screen.

12. The system according to claim 1, wherein at least one of the two or more models relates to at least one of a fluid injection aspect of the subject imaging study and an image creation aspect of the subject imaging study.

13. The system according to claim 12, wherein at least two of the two or more models relate to at least one of the fluid injection aspect of the subject imaging study and the image creation aspect of the subject imaging study.

14. The system according to claim 13, wherein a first of the two or more models relates to the fluid injection aspect of the subject imaging study and a second of the two or more models relates to the image creation aspect of the subject imaging study.

15. The system according to claim 1, wherein the parameters include at least one of the following: total contrast volume, maximum flow rate, contrast delivery rate, average flow rate, contrast temperature, contrast viscosity, contrast concentration, IV access location, region of scan, potential applied to X-ray tube, maximum current applied to X-ray tube, scan speed, scan duration, radiation dose, signal/noise ratio, contrast/noise ratio, or spatial/resolution ratio.

16. The system according to claim 1, wherein the information about the subject patient comprises at least one of height, weight, body mass index, cardiac output, gender, age, ethnicity, thoracic width, thoracic circumference, medications taken, underlying medical conditions, physical ability, vital signs, pregnant/expecting to become pregnant, genetic predisposition of the subject patient, allergies, results of previous imaging exams for the subject patient, and known radiation sensitivity of the subject patient.

17. The system according to claim 1, wherein the one or more data sources comprise at least one of an electronic medical record (EMR) system comprising an electronic medical record of the patient, an electronic health record (EHR) system, a patient procedure tracking system, a radiology analytics system (RAS), a digital pathology system (DPS), a picture archive and communication system (PACS), a hospital data system, a hospital purchase order system comprising an order for a study to be performed for the subject patient, a database comprising previous scan results for the patient, a database comprising previous scan results for one or more other patients, or a government guidelines database of acceptable radiation dose and contrast dose levels.

18. The system according to claim 1, wherein the information about the subject imaging study comprises information about a fluid injector associated with the subject imaging study wherein the information about the fluid injector includes information from a test injection or patency check using saline, information about capabilities and tolerances of the fluid injector, and/or presence of external sensors for monitoring injections performed by the fluid injector.

19. The system according to claim 1, wherein the one or more risk factors are related to at least one of contrast dose, radiation dose, risk of extravasation, patient discomfort, risk of anaphylactic shock, and image quality.

20. The system according to claim 6, wherein the one or more iteration cycles optimize one or more parameters of the baseline study protocol by applying an algorithm that minimizes or maximizes selected parameter values, an algorithm for ensuring that certain parameters are within a target or threshold range, or a weighted function to parameter values of the baseline study protocol.

21. A method for generating protocols that can be used for medical imaging studies, comprising:
receiving, from one or more data sources, information about a subject patient and information about a subject imaging study;
determining, based on the information about the subject patient and the information about the subject imaging study, one or more risk factors particular to the subject patient;
selecting two or more models, wherein the two or more models comprise at least one model of each of at least two aspects of the subject imaging study; and
applying the two or more models to generate a baseline study protocol for the subject imaging study, wherein the baseline study protocol is based upon at least the information about the subject patient and the one or more risk factors, and wherein the baseline study protocol comprises parameters of the at least two aspects of the subject imaging study.

22-41. (canceled)
